(19) United States
(12) Patent Application Publication
(10) Pub. No.: US 2019/0060191 A1
(43) Pub. Date: Feb. 28, 2019

(54) UV-PROTECTIVE COMPOSITIONS AND THEIR USE

(71) Applicant: LANDA LABS (2012) LTD., Rehovot (IL)

(72) Inventors: Benzion LANDA, Nes Ziona (IL); Sagi ABRAMOVICH, Ra'anana (IL); Snir DOR, Petach Tikva (IL)

(21) Appl. No.: 16/154,109

(22) Filed: Oct. 8, 2018

Related U.S. Application Data
(63) Continuation-in-part of application No. PCT/IB2017/051975, filed on Apr. 6, 2017.
Application No. PCT/IB2017/051975 is a continuation-in-part of application No. 16/091,539.

(30) Foreign Application Priority Data
Apr. 6, 2016 (GB) 1605857.0

(51) Int. Cl. A61K 8/29; A61K 8/02; A61K 8/81; A61Q 17/04

(52) U.S. Cl. CPC A61K 8/29; A61K 8/0245; A61K 8/0254; A61K 2800/412; A61K 8/8135; A61Q 17/04; A61K 2800/413; A61K 8/8147

(57) ABSTRACT

Disclosed are UV-protective compositions comprising BLT crystals having the formula Bi₍₄₋ₓ₎La₍ₓ₎Ti_{(3-y)}Fe_{(y)}O₁₂, wherein x is between 0.1 and 1.5; and wherein y is between 0.01 and 2. There are also disclosed compositions comprising nanoparticles of such BLT crystals, the nanoparticles being optionally dispersed in a polymer matrix. Methods of preparation and uses of such compositions are also provided.

### CROSS-REFERENCE TO RELATED APPLICATIONS

This application is a Continuation-In-Part (CIP) of International Application No. PCT/IB2017/051975, filed Apr. 6, 2017, which claims priority from patent application GB1605857.0, filed Apr. 6, 2016. This patent application is also a Continuation-In-Part (CIP) of U.S. patent application Ser. No. 16/091,539 which is a 371 national stage filing of International Application No. PCT/IB2017/051975. All of the aforementioned applications are incorporated herein by reference for all purposes as if fully set forth herein.

### FIELD

The present disclosure relates to the field of protection from ultraviolet radiation, and more particularly, to UV-protective compositions comprising lanthanum-modified bismuth titanate (BLT) crystals, neat or polymer-embedded, to methods for preparing the same and uses thereof.

### BACKGROUND

Ultraviolet (UV) radiation is ubiquitous, the sun being the most common source of UV radiation, although not the only source. As UV radiation can cause damage to people, animals and objects, compositions that provide protection from UV radiation are useful.

In the biological context, UV-protective compositions, i.e. compositions that reduce or block the transmission of UV rays, are commonly employed to protect against sunburn. Sunburn is a form of radiation burn resulting from overexposure to UV radiation, typically from the sun, but also from artificial sources, such as tanning lamps, welding arcs, and ultraviolet germicidal irradiation.

Normal symptoms of sunburn in humans and other animals include reddening of the skin, general fatigue and mild dizziness. An excess of UV radiation can be life-threatening in extreme cases. Excessive UV radiation is considered to be the leading cause of non-malignant skin tumors, and also increases the risk of certain types of skin cancer.

Sunscreen compositions comprising UV-protective agents are commonly used to prevent sunburn, and are believed to reduce the incidence of squamous cell carcinomas and melanomas. Furthermore, they have been reported to delay the development of wrinkles and additional age-related and exposure-related skin conditions.

Specifically, sunscreen compositions are topical compositions that include UV-protecting agents that absorb and/or reflect at least some of the sun's UV radiation on areas of skin exposed to sunlight, and thus reduce the effect of UV radiation on the skin. Depending on their mode of action, they are typically classified as chemical or physical sunscreens.

Chemical sunscreen compositions comprise organic compounds that absorb UV radiation to reduce the amount of UV radiation that reaches the skin. Being transparent to visible light and thereby being invisible when applied to the skin, chemical sunscreen compositions are popular for use. However, some organic compounds used in chemical sunscreen compositions have been found to generate free radicals that may cause skin damage, irritation and accelerated aging of the skin. Furthermore, organic materials may be absorbed into the skin, resulting in long-term detrimental health effects. Chemical sunscreen compositions may require the addition of a photostabilizer. Another possible drawback when using organic UV-protecting agents in compositions protecting the surfaces of inanimate objects, is that they tend to develop a yellowish tone with time and with exposure to radiation.

Physical sunscreen compositions reflect and/or absorb UV radiation. Known physical sunscreen compositions comprise particles of inorganic materials, mainly titanium oxide and/or zinc oxide. In order to obtain absorption and/or reflection of ultraviolet radiation over the full UVA and UVB range, relatively large particles are used. Due to the large particle size, however, such sunscreen compositions are opaque and tend to leave a white film on the skin.

Many sunscreen compositions protect against sunburn-causing UV radiation in the 280-315 nm range (UVB radiation), but do not protect against UV radiation in the 315-400 nm range (UVA radiation), which may not be the primary cause of sunburn, but can increase the incidence of melanoma and photodermatitis. Protection against UVA radiation is an important factor for cosmetic or medical products for humans, but less important for UV-protecting compositions considered for surface coatings of inanimate objects, for which UVB radiation is the leading cause of damage.

It is generally preferred that sunscreen compositions, when applied to the skin, are transparent to the eye. In order for physical sunscreen compositions to be transparent, the particles of inorganic material should be in the form of nanoparticles, which absorb and/or scatter UV light but not visible light, rendering the nanoparticles substantially transparent to the eye when applied to the skin. However, use of nanoparticles reduces the range of wavelengths absorbed by the inorganic materials. Some known sunscreen compositions therefore block both UVA and UVB radiation by use of a combination of different UV-absorbing or scattering materials, generally termed UV-protecting agents, each of which blocks radiation over a limited range of the UV spectrum.

Similarly, UV-protective compositions can benefit inanimate materials or objects that may be negatively affected by UV radiation. For instance, UV radiation can reduce the life-span of materials (e.g., natural and synthetic polymers), and may modify colors of objects, especially in articles that are subjected to prolonged sun exposure, such as buildings or vehicles.

Various coatings are known to provide protection against UV radiation damage by blocking or reducing transmission of UV rays. Use of such coatings may reduce the detrimental effect of UV radiation on living animals. For example, the use of such a coating on optical lenses may reduce the transmission of UV radiation, thereby reducing the incidence of UV-induced optical disorders such as cataracts. Materials serving for the fabrication of windows incorporating or coated with suitable UV-protecting agents may reduce the transmission of UV radiation to subjects, plants, surfaces or objects shielded by such windows. Applicant has disclosed sunscreen compositions comprising inorganic nanoparticles, inter alia in PCT Publication Nos. WO 2016/151537 and WO 2017/013633.

The present inventors have recognized a need for improved UV-protective compositions containing UV-protective nano-particulate materials, methods of production thereof, and UV-protective articles of manufacture containing such UV-protective nano-particulate materials.

### SUMMARY

The present disclosure, in at least some embodiments thereof, provides ultraviolet radiation protective compositions, such as, sunscreen compositions, that, when applied to a surface, provide protection from UV radiation, which in some embodiments have a broad-spectrum UV-protective activity, such compositions comprising Lanthanum-Modified Bismuth Titanate (BLT) crystals, optionally doped by iron (Fe) atoms.

According to an aspect of some embodiments, there is provided a UV-protective composition comprising one or more Lanthanum-Modified Bismuth Titanate (BLT) crystals each independently having the chemical formula Bi₍₄₋ₓ₎La₍ₓ₎Ti_{(3-y)}Fe_{(y)}O₁₂ as an ultraviolet-absorbing agent, wherein x is between 0.1 and 1.5; and wherein y is between 0 and 2.

The doped (i.e., y>0) or undoped (i.e., y=0) BLT crystals are a composite material, having properties which differ from those individually characterizing their constituting starting compounds. One or more crystals, of the same or different general chemical formula, may form particles or nanoparticles as described below.

The Lanthanum-Modified Bismuth Titanate crystals can be synthesized using different ratios of Bismuth Trioxide (Bi₂O₃; also referred to as Bismuth(III) Oxide or simply Bismuth Oxide), Titanium Dioxide (TiO₂; often referred to as Titanate or Titanium Oxide) and Lanthanum Oxide (La₂O₃) by a variety of methods readily known to the person skilled in the art of preparing such composite materials. One such method shall be detailed herein-below.

For conciseness, the mixture of the individual metal oxide constituents shall be referred to as BLTO, whereas the crystal as prepared, comprising the composite material, shall be termed hereinafter BLT, such acronyms eventually followed by the ratio between at least two of the constituents. The ratio is typically provided on a molar basis, but may also be provided on a weight per weight basis. As used herein, the term “BLT” includes both the doped and the undoped crystal.

In the event that iron atoms (as available for instance from Iron(III) Oxide or Ferric Oxide (Fe₂O₃)) optionally substitute atoms of the composite material, typically Titanium, the so-called “doped” crystal is formed. In such case, the crystal acronym for the chemical formula may eventually be followed by the molar ratio of substitution between the iron substituent and the atom being replaced. For instance, BLT Fe:Ti 1:2 refers to a Lanthanum-Modified Bismuth Titanate composite material wherein 1 mole of Ferric Oxide (Fe₂O₃) is included in the synthetic process for every 2 moles of Titanium Oxide (TiO₂). BLTO Fe:Ti 1:2 refers to same amounts of metal oxide constituents, including the Ferric Oxide intended for substitution, however the compounds are only mixed and not further processed for the preparation of the previously described composite material and resulting crystal.

According to an aspect of some embodiments, there is provided a UV-protective composition comprising one or more Lanthanum-Modified Bismuth Titanate (BLT) crystals each independently having the chemical formula Bi₍₄₋ₓ₎La₍ₓ₎Ti_{(3-y)}Fe_{(y)}O₁₂ as an ultraviolet-absorbing agent, wherein x is between 0.1 and 1.5; and wherein y is between 0 and 2.

In some embodiments, the doped or undoped BLT crystals have a perovskite structure.

In particular embodiments, x is between 0.5 and 1.0, or between 0.7 and 0.8. In some embodiments, x is at least 0.2, at least 0.4, or at least 0.6. In other embodiments, x is at most 1.2, at most 1.0, at most 0.9, or at most 0.8.

In some embodiments, y is greater than zero, in which case crystals having the chemical formula Bi₍₄₋ₓ₎La₍ₓ₎Ti_{(3-y)}Fe_{(y)}O₁₂ can also be referred to as Fe-doped BLT crystals. In particular embodiments, y is between 0.01 and 2, between 0.01 and 1.8, between 0.1 and 1.6, between 0.2 and 1.5, between 0.3 and 1.3, between 0.5 and 1, between 0.12 and 2, between 0.25 and 2, between 0.25 and 1.8, or between 0.5 and 1.7. In some embodiments, y is at least 0.1, at least 0.2, at least 0.3, at least 0.4, at least 0.5, at least 0.6, or at least 0.7. In other embodiments, y is at most 1.2, at most 1.0, at most 0.9, or at most 0.8.

In other embodiments, y equals zero and in such case a BLT crystal can also be referred to as an undoped BLT crystal, having the simplified chemical formula Bi₍₄₋ₓ₎La₍ₓ₎Ti₃O₁₂.

In some embodiments, the molar ratios of Fe to Ti are respectively selected from (0.0625:2.9375), (0.125:2.875), (0.25:2.75), (1:2) and (1.5:1.5).

It can be appreciated that the level of Fe-doping may modify the spectrum of absorbance of BLT crystals. For instance, when a higher level of Fe-doping is implemented (i.e., y is closer to 2), the absorbance of the doped BLT is shifted towards the visible range, whereas a lower level of Fe-doping (i.e., y is closer to 0.01, e.g., having a value of less than 0.5 or less than 0.25), provides for predominant absorbance in the UV-range.

Compositions including BLT crystals having a higher level of Fe-doping can be visible to the human eye, having a tint. The desired level of Fe-doping can be determined according to the purpose and usage of the UV-protective compositions. For example, if transparency is desired, e.g., for cosmetic purposes on faint skin or for clear lacquers, a lower level of Fe-doping is preferred. For cosmetic compositions or coatings of inanimate objects being anyhow tinted, higher Fe-doping can be tolerated or even desired, as long as compatible with the intended color of the composition.

Moreover, the amount of Fe-doped BLT crystals and nanoparticles within the final composition may also affect the desired or tolerable level of doping. A UV-protective composition comprising a relatively low amount of doped BLT may permit the use of nanoparticles having a relatively higher level of Fe-doping, as the tinting such added doping may provide would be attenuated by the low concentration of the composite material.

Additionally, as the size of the nanoparticles affect their absorbance over the spectrum, at a given concentration of particles within a final UV-protective composition, smaller particles having a higher level of Fe-doping may behave similarly as larger particles having a lower level of Fe-doping.

Therefore, the extent of Fe-doping that can be satisfactory for a particular UV-protective composition depends inter alia on the intended use of the composition, the size of the nanoparticles of Fe-doped BLT crystals and the concentration of the nanoparticles within the composition.

The compositions described herein are for use in both living subjects and inanimate objects (e.g., UV-protective coating of articles routinely exposed to UV radiation).

Therefore, some embodiments of the present disclosure relate to compositions providing protection against ultraviolet radiation (i.e. UV-protective compositions), and more particularly, to UV-protective compositions comprising BLT crystals, optionally doped by iron atoms, as an ultraviolet-absorbing agent.

In some embodiments, the doped or undoped BLT crystals are present in the composition as discrete, individual nanoparticles consisting of one or more said crystals, at least 50% of the total number of said nanoparticles having at least one dimension (e.g., as determined by microscopy such as HRSEM or STEM, or a hydrodynamic diameter such as a DLS-determined hydrodynamic diameter) of up to about 500 nm, up to about 400 nm, or up to about 300 nm. In some embodiments, at least 50% of the total number of said nanoparticles have at least one dimension of up to about 250 nm, up to about 200 nm, up to about 150 nm, or up to about 100 nm. In some such embodiments, the nanoparticles consist of crystals having the same chemical formula.

In some embodiments, the doped or undoped BLT crystals are present in the composition as discrete, individual nanoparticles consisting of one or more said crystals, at least 50% of the total volume of said nanoparticles having at least one dimension of up to about 500 nm, or up to about 400 nm, or up to about 300 nm. In some embodiments, at least 50% of the total volume of said nanoparticles have at least one dimension of up to about 250 nm, up to about 200 nm, up to about 150 nm, or up to about 100 nm.

Without wishing to be bound by a particular theory, it is believed that when at least 50% of the total number or volume of nanoparticles have at least one dimension in the range of between about 250 nm and about 500 nm, scattering of incident light may occur, resulting in the compositions containing such nanoparticles being visible to the human eye. This can be suitable when transparency is not an essential feature of the desired product for which said compositions are used, e.g., UV-protective coatings for outdoor furniture or for sunglasses lenses. For UV-protective compositions wherein light scattering is to be avoided, at least 50% of the total number or volume of the nanoparticles of BLT crystals shall preferably have their dimensions in a range not exceeding 250 nm.

In some embodiments, at least 55%, at least 60%, at least 65%, at least 70%, at least 80%, or at least 85% of the total number or total volume of nanoparticles has at least one dimension of up to about 500 nm, up to about 400 nm, or up to about 300 nm. In some embodiments, at least 55%, at least 60%, at least 65%, at least 70%, at least 80%, or at least 85% of the total number or total volume of nanoparticles has at least one dimension of up to about 250 nm, up to about 200 nm, up to about 150 nm, or up to about 100 nm. In some such embodiments, the nanoparticles consist of crystals having the same chemical formula.

In some embodiments, at least 90%, or at least 95%, or at least 97.5%, or at least 99% of the total number or total volume of nanoparticles of the doped or undoped BLT crystals present in the composition has a hydrodynamic diameter of up to about 500 nm, up to about 400 nm, or up to about 300 nm. In some embodiments, at least 90%, or at least 95%, or at least 97.5%, or at least 99% of the total number or total volume of nanoparticles of the doped or undoped BLT crystals present in the composition has a hydrodynamic diameter of up to about 250 nm, up to about 200 nm, up to 150 nm, or up to about 100 nm.

According to an aspect of the invention, there is provided a UV-protective composition comprising Fe-doped lanthanum-modified bismuth titanate (BLT) crystals each independently having the chemical formula Bi₍₄₋ₓ₎La₍ₓ₎Ti_{(3-y)}Fe_{(y)}O₁₂ as an ultraviolet-absorbing agent, wherein x is between 0.1 and 1.5; wherein y is at least 0.01 and at most 2, the BLT crystals forming discrete nanoparticles, wherein at least 50% of a total number of said discrete nanoparticles have at least one dimension (e.g., as determined by microscopy such as HRSEM or STEM, or a hydrodynamic diameter such as a DLS-determined hydrodynamic diameter) of up to 250 nm.

In some embodiments, the doped or undoped BLT nanoparticles are present in the UV-protective composition dispersed with a dispersant, optionally in the presence of a carrier. Without wishing to be bound by any particular theory, the dispersant can serve as a stabilizer, keeping the individual nanoparticles discrete, separated and well-dispersed in the composition.

In some embodiments, the dispersant is present in the composition in an amount sufficient for maintaining the nanoparticles of BLT crystals homogeneously dispersed for the lifespan of the UV-protective composition. In some case, it will be recommended to shake, stir or otherwise agitate the composition ahead of use to restore homogenous dispersion of the nanoparticles.

In some embodiments, the composition contains at least 30% weight per weight percentage (wt. %) of dispersant per weight of the nanoparticles, at least 40 wt. %, or at least 50 wt. %. In some embodiments, the composition contains at most 70 wt. % of dispersant per weight of the nanoparticles (or per total weight of the composition), at most 65 wt. %, or at most 60 wt. %. In some embodiments, a dispersant, when present, is in the range of 30 wt. % to 70 wt. % per weight of the nanoparticles (or per total weight of the composition), in the range of 33 wt. % to 66 wt. %, or in the range of 40 wt. % to 60 wt. %.

In a particular embodiment, the weight per weight ratio of the doped or undoped BLT nanoparticles and the dispersant is between 2:1 and 1:2.

In some embodiments, the doped or undoped BLT nanoparticles are present in the composition dispersed in a polymer matrix. In particular embodiments the nanoparticles of the composite UV-absorbing agent are dispersed in the polymer matrix in presence of a dispersant, the polymer matrix being in an oil-based or a water-based carrier.

As used herein, an oil-based carrier or vehicle relates to a material (or a mixture of materials) which has a low to substantially null miscibility in water, 5% of the weight of the material or less being water miscible. In some embodiments, less than 4 wt. % of the oil-based carrier, less than 3 wt. %, less than 2 wt. % or less than 1 wt. % can dissolve in water. In contrast, a water-based carrier or vehicle (which may contain for instance at least 50 wt. % water) relates to a material (or a mixture of materials) which has a high to substantially full miscibility in water, 5% of the weight of the material or less being water immiscible. In some embodiments, less than 4 wt. % of the water-based carrier, less than 3 wt. %, less than 2 wt. % or less than 1 wt. % cannot dissolve in water.

In some embodiments, the composition contains less than 5 weight per weight percentage (wt. %), less than 4 wt. %, less than 3 wt. %, less than 2 wt. %, less than 1 wt. %, less than 0.5 wt. %, less than 0.1 wt. % or less than 0.05 wt. % organic ultraviolet-absorbing agent(s). Suitably the composition is generally devoid of an organic ultraviolet-absorbing agent. Typically, the composition is free of an organic ultraviolet-absorbing agent.

In some embodiments, the composition contains less than 5 wt. %, less than 4 wt. %, less than 3 wt. %, less than 2 wt. %, less than 1 wt. %, less than 0.5 wt. %, less than 0.1 wt. % or less than 0.05 wt. % additional inorganic ultraviolet-absorbing agent(s). Suitably the composition is generally devoid of an additional inorganic ultraviolet-absorbing agent. Typically, the composition is free of an additional inorganic ultraviolet-absorbing agent. In some embodiments, the one or more doped or undoped BLT crystals constitute the only ultraviolet-absorbing agents in the composition.

In some embodiments, the doped or undoped BLT crystals are present in the composition in the form of nanoparticles at a concentration in the range of from about 0.001 wt. % to about 40 wt. % of the total composition.

In some embodiments, the composition further comprises silver particles.

In some embodiments, the silver particles comprise silver nanoparticles having at least one dimension of up to about 50 nm.

In some embodiments, at least 90%, at least 95%, at least 97.5% or at least 99% of the number of silver nanoparticles present in the composition has at least one dimension of up to about 50 nm.

In some embodiments, at least 90%, at least 95%, at least 97.5% or at least 99% of the volume of silver nanoparticles present in the composition has at least one dimension of up to about 50 nm. In some embodiments, wherein the composition comprises silver nanoparticles, the composition is devoid of an additional ultraviolet-absorbing agent.

In some embodiments, the silver particles are present in the composition at a concentration in the range of from about 0.01 wt. % to about 10 wt. % of the total composition.

In some embodiments, the composition further comprises one or more of a carrier, an excipient, an additive and combinations thereof. Carriers, excipients and additives being cosmetically acceptable are preferred for use in living subjects, but may not be required for use on the surfaces of inanimate objects.

In some embodiments, the composition is in a form selected from the group consisting of an aerosol, a cream, an emulsion, a gel, a lotion, a mousse, a paste, a liquid coat or a spray.

In some embodiments, the composition is formulated as one of the following: (a) a skin-care composition for application to human or non-human animal skin; (b) a hair-care composition for application to human or non-human animal hair; or (c) a coating composition for application to an inanimate surface.

In a further aspect, embodiments of the present disclosure provide use of afore-described doped or undoped BLT crystals for the preparation of a composition for protecting a target surface, such as a surface of a living subject and/or an inanimate object, against a harmful effect of UV radiation. The compositions, comprising an efficacious amount of BLT, can be formulated as suitable for application upon the intended surfaces, such preparations being known to persons skilled in the relevant formulations.

According to one embodiment, there is provided a composition as described herein, for use in protecting a subject against a harmful effect of UV radiation

According to one embodiment, there is provided a composition as described herein, for use in protecting the skin of a subject against a harmful effect of UV radiation. In some such embodiments, the composition is in the form of a topical composition. In such embodiments, the composition can be in any form suitable to skin-care products, such as facial-care products, make-up products, body-care products, hand-care products and/or foot-care products. Such skin-care products can be applied to the skin of a subject by any conventional method and/or for any duration of time that need not be detailed herein.

According to a further embodiment, there is provided a composition as described herein, for use in protecting the hair of a subject against a harmful effect of UV radiation. In some such embodiments, the composition is in the form of a hair-care product, such as a hair-care product selected from the group consisting of a shampoo, a conditioner and a hair mask. Such hair-care products can be applied to the hair of a subject by any conventional method and/or for any duration of time that need not be detailed herein.

In some embodiments of a use of the composition, the subject is a human subject. In alternative embodiments of a use of the composition, the subject is a non-human animal.

In some embodiments of the use of the composition, the target surface is a surface of an inanimate object, such as, for example, an object, or a material. In some such embodiments, the composition is in the form of a coating, including liquid coatings, such as a varnish, a lacquer or an emulsion, and non-liquid coatings, such as a paste, a gel, or a mousse. Though UV-protective compositions applicable to the surfaces of inanimate objects are herein referred to as “coatings”, it will be readily understood that such compositions may also permeate, impregnate or be otherwise embedded at least to some extent within the surfaces of the objects being protected. Such coating products can be applied to the surface of an inanimate object by any conventional method that need not be detailed herein.

In some embodiments, protecting against ultraviolet radiation comprises protecting against a harmful effect of ultraviolet B radiation. In some embodiments, protecting against ultraviolet radiation comprises protecting against a harmful effect of ultraviolet A radiation and ultraviolet B radiation.

In some embodiments, the composition has a critical wavelength of at least 370 nm, or at least 371 nm, or at least 372 nm, or at least 373 nm, or at least 374 nm, or at least 375 nm, or at least 376 nm, or at least 377 nm, or at least 378 nm, or at least 379 nm, or at least 380 nm, or at least 381 nm, or at least 382 nm, or at least 383 nm, or at least 384 nm, or at least 385 nm, or at least 386 nm, or at least 387 nm, or at least 388 nm, or at least 389 nm, or at least 390 nm, or at least 391 nm, or at least 392 nm. In some embodiments, the composition has a critical wavelength of at most 400 nm, or at most 399 nm, or at most 398 nm, or at most 397 nm, or at most 396 nm, or at most 395 nm, or at most 394 nm, or at most 393. In particular embodiments, the composition has a critical wavelength in the range between 370 nm and 400 nm, between 375 nm and 400 nm, between 380 nm and 399 nm, or between 385 and 398 nm.

In some embodiments, the area under the curve (AUC) formed by the UV-absorption of the one or more BLT crystals as a function of wavelength in the range of 280 nm to 400 nm (AUC₂₈₀₋₄₀₀) is at least 75%, at least 85% or at least 95% of the AUC formed by the same crystals at the same concentration in the range of 280 nm to 700 nm (AUC₂₈₀₋₇₀₀).

In another aspect of the disclosure, there is provided a method of manufacturing nanoparticles of doped or undoped BLT crystals as herein described, the composites of the BLT crystals being present in any desired stochiometric amount. The method comprises:
a) providing doped or undoped BLT particles, wherein at least 50% of the total number of said particles have at least one dimension not exceeding 1 mm;
b) combining the doped or undoped BLT particles with a dispersant, optionally in the presence of a carrier, to obtain a slurry; and
c) milling the slurry of step b) to obtain nanoparticles of doped or undoped BLT crystals, the nanoparticles having at least one dimension not exceeding 500 nm.

In some embodiments, the milling of step b) is high-energy milling

In some embodiments, the doped or undoped BLT particles provided in step a) may be prepared by any method known in the art. One such method includes:
i. mixing together powders of lanthanum, bismuth and titanium, each independently in the form of metal oxides, metal nitrates or metal carbonates, in appropriate ratios (so as to obtain the desired stoichiometric amount), to obtain a mixed or substantially homogeneous mixture;
ii. calcinating the mixture of step (i) at at least one calcinating temperature, to obtain doped or undoped BLT crystals or agglomerates thereof;
iii. milling the doped or undoped BLT crystals or agglomerates thereof, so as to obtain doped or undoped BLT particles, wherein at least 50% of the total number of said particles have at least one dimension not exceeding 1 mm.

The lanthanum, bismuth and titanium, each of which may be in the form of metal oxides, metal nitrates or metal carbonates, can be referred to as a metal starting material.

When Fe-doped BLT is desired, an amount of ferric oxide, ferric carbonate or ferric nitrate, selected to provide the intended doping ratio, is combined with the other metal starting materials, and the corresponding amount of the titanium starting material is reduced accordingly.

In some embodiments, the milling of step iii) is low-energy milling.

In some embodiments, prior to the milling, the doped or undoped BLT crystals or agglomerates thereof obtained in step ii), are cooled, or allowed to cool, to a temperature of at most 150° C., at most 100° C., at most 70° C., at most 50° C., or at most to an ambient temperature (circa 23° C.).

In some embodiments, the dispersant in step b) is added in an amount and a form that is sufficient to suspend the nanoparticles, such that the dispersant also serves as a carrier. For instance, the dispersant is in liquid form. In particular embodiments, a dedicated carrier is added in addition to the dispersant in step b).

In some embodiments, the choice of the dispersant and optional carrier for the manufacturing of the nanoparticles of doped or undoped BLT crystals depends on the further processing of the nanoparticles for the preparation of the UV-protective compositions, and their intended use.

When the nanoparticles are to be dispersed in a composition including an oil-based vehicle, selection of an oil-based carrier and dispersant compatible with such oil-based carrier and/or vehicle can be done as early as the stage of manufacturing of the nanoparticles. When the nanoparticles are to be dispersed in a composition including a water-based vehicle, then a water-based carrier and dispersant compatible with such water-based carrier and/or vehicle can be selected.

In some embodiments, the dispersant used in the preparation of the nanoparticles is oleic acid, a polyhydroxystearic acid (such as commercially available from Innospec Performance Chemicals under tradenames Dispersun DSP-OL100 and DSP-OL300 or from Phoenix Chemicals as Pelemol® PHS-8) or polyacrylic acid and salts thereof, e.g. sodium salt (PAA, such as commercially available from Sigma Aldrich, USA, under the CAS nos. 9003-01-4, for the acid form, and 9003-04-7, for the sodium salt form).

According to a further aspect of some embodiments of the disclosure, there is provided a method of manufacturing a UV-protective composition, comprising combining doped or undoped BLT crystals, as an ultraviolet-absorbing agent, with other ingredients in proportions and in a manner suitable to make a UV-protective composition as described herein. In some embodiments, the UV-protective composition is manufactured and formulated as a sunscreen composition for application to skin or hair of a human or non-human living subject. In some embodiments, the composition is manufactured and formulated as a composition for application to a surface of an inanimate object.

There is also provided, in accordance with an embodiment of the invention, a method of protecting a surface from UV radiation, which comprises applying to a surface in need of such protection a UV-protective composition in an amount sufficient to achieve such protection, said UV-protective composition comprising Fe-doped lanthanum-modified bismuth titanate (BLT) crystals each independently having the chemical formula Bi₍₄₋ₓ₎La₍ₓ₎Ti_{(3-y)}Fe_{(y)}O₁₂ as an ultraviolet-absorbing agent, wherein x is between 0.1 and 1.5; wherein y is at least 0.01 and at most 2, the BLT crystals forming discrete nanoparticles, wherein at least 50% of a total number of said discrete nanoparticles have at least one dimension of up to 250 nm.

In some embodiments, the surface is human skin. In some embodiments, the surface is non-human skin, i.e. animal skin. In some embodiments, the surface is hair. In some embodiments, the hair is human hair. In some embodiments, the hair is non-human hair, i.e. animal hair. In some embodiments, the surface is a surface of an inanimate object.

According to another aspect of the invention, there is provided an article covered or coated with the UV-protective composition, comprising the doped or undoped BLT crystals.

As used herein, the term “nanoparticles” refers to particles of any suitable shape, which may consist of one or more crystals as herein disclosed, wherein the size of at least one dimension is 250 nm or less or 200 nm or less, hereinafter also referred to as the smallest dimension, and wherein a greatest size in a different dimension of the particles, also termed a greatest dimension, is of no more than about 500 nm.

For example, in some embodiments where the particles have a flake-like shape, the smallest dimension of the nanoparticles can be their thickness which can be of up to about 250 nm or 200 nm, while their length can be of no more than about 500 nm.

For example, in some embodiments where the particles have a rod-like shape, their cross section along their longitudinal axis could be approximated to ellipsoids having at least their minor axis constituting a smallest dimension of no more than about 250 nm or 200 nm and the length of the rods being no more than about 500 nm.

For example, in some embodiments where the particles have a sphere-like shape that could be approximated by three diameters one for each of the X-, Y- and Z-direction, at least one of the three diameters is not more than about 250 nm or 200 nm and a greatest of the three diameters can be no more than about 500 nm.

In some embodiments, the smallest dimension of the nanoparticles is not more than about 180 nm, not more than about 160 nm, not more than about 140 nm, not more than about 120 nm, or not more than about 100 nm.

In some embodiments, the smallest dimension of the nanoparticles is at least about 10 nm, at least about 15 nm or at least about 20 nm.

In some embodiments, the greatest dimension of the nanoparticles is not more than about 400 nm, not more than about 300 nm, not more than about 200 nm, or not more than about 150 nm.

In some embodiments, the nanoparticles of BLT and/or the compositions including the BLT crystals disclosed herein are substantially invisible to the human eye, in particular when applied to a subject and more particularly when applied on a pale skin on which a tint of the composition could be detected and undesired.

In some embodiments, the compositions are visible to the human eye when applied to a subject. In some such embodiments, iron doped BLT provides a reddish color that is beneficial in the preparation of a product in which such color is desirable, e.g. a make-up product such as a blusher, or a tinted coating for application to a surface of an inanimate object.

In some embodiments, the size of the particles (e.g., BLT nanoparticles or matrix elements or flakes optionally embedding them) is determined by microscopy techniques, as known in the art.

In some embodiments, the size of the particles is determined by Dynamic Light Scattering (DLS). In DLS techniques the particles are approximated to spheres of equivalent behavior and the size can be provided in term of hydrodynamic diameter. DLS also allows assessing the size distribution of a population of particles.

Distribution results can be expressed in terms of the hydrodynamic diameter for a given percentage of the cumulative particle size distribution, either in terms of numbers of particles or in terms of particle volume, and are typically provided for 10%, 50% and 90% of the cumulative particle size distribution. For instance, D50 refers to the maximum hydrodynamic diameter below which 50% of the sample volume or number of particles, as the case may be, exists and is interchangeably termed the median diameter per volume (D_{V}50) or per number (D_{N}50), respectively.

In some embodiments, the nanoparticles of the disclosure have a cumulative particle size distribution of D90 of 500 nm or less, or a D95 of 500 nm or less, or a D97.5 of 500 nm or less or a D99 of 500 nm or less, i.e. 90%, 95%, 97.5% or 99% of the sample volume or number of particles respectively, have a hydrodynamic diameter of no greater than 500 nm.

In some embodiments, the nanoparticles of the disclosure have a cumulative particle size distribution of D90 of 250 nm or less, or a D95 of 250 nm or less, or a D97.5 of 250 nm or less or a D99 of 250 nm or less, i.e. 90%, 95%, 97.5% or 99% of the sample volume or number of particles respectively, have a hydrodynamic diameter of no greater than 250 nm.

In some embodiments, the nanoparticles of the disclosure have a cumulative particle size distribution of D90 of 200 nm or less, or a D95 of 200 nm or less, or a D97.5 of 200 nm or less or a D99 of 200 nm or less, i.e. 90%, 95%, 97.5% or 99% of the sample volume or number of particles respectively, have a hydrodynamic diameter of no greater than 200 nm.

In some embodiments, the cumulative particle size distribution of the population of nanoparticles is assessed in term of number of particles (denoted D_{N}) or in term of volume of the sample (denoted D_{V}) comprising particles having a given hydrodynamic diameter.

Any hydrodynamic diameter having a cumulative particle size distribution of 90% or 95% or 97.5% or 99% of the particles population, whether in terms of number of particles or volume of sample, may be referred to hereinafter as the “maximum diameter”, i.e. the maximum hydrodynamic diameter of particles present in the population at the respective cumulative size distribution.

It is to be understood that the term “maximum diameter” is not intended to limit the scope of the present teachings to nanoparticles having a perfect spherical shape. This term as used herein encompasses any representative dimension of the particles at cumulative particle size distribution of at least 90%, e.g., 90%, 95%, 97.5% or 99%, or any other intermediate value, of the distribution of the population.

Dimensions of particles can also be assessed (or confirmed) by microscopy (e.g., light microscopy, confocal microscopy, SEM, STEM, etc.). Such techniques may be more suitable than DLS for particles (such as matrix flakes) having non-globular shapes. The particles may be characterized by an aspect ratio, e.g., a dimensionless ratio between the smallest dimension of the particle and the longest dimension or equivalent diameter in the largest plane orthogonal to the smallest dimension, as relevant to their shape. The equivalent diameter (Deq) is defined by the arithmetical average between the longest and shortest dimensions of that largest orthogonal plane. Particles having an almost spherical shape are characterized by an aspect ratio of approximately 1:1, whereas flake-like particles, such as matrix flakes, can have an aspect ratio of up to 1:100, or even more.

As readily appreciated by a person skilled in measurement of particle size, combining a variety of techniques also allows to assess whether the particles or nanoparticles are individuals or agglomerates, and whether they would or not be well-dispersed within their respective media.

As further detailed herein-below, nanoparticles of BLT crystals can in some embodiments be immobilised within a polymer matrix. The matrix can form distinct elements, which may assume a variety of shapes. For topical application, a platelet shape of polymer matrix element is deemed particularly suitable, as the platelets may lay flat on the skin when applied, providing a better coverage than, e.g., sphere-shaped particles. Such flat platelets of polymers can also be advantageous for industrial use, e.g., for electrostatic coatings. Such matrix flakes can be characterized by a flake length (Lf, the longest dimension in the plane of the flake), a flake width (Wf, the largest dimension in the plane of the flake, such width being transverse to the length), and a flake thickness (Tf, the largest thickness being measured orthogonally to the plane in which the length and width of the flake are defined), such that Tf is smaller than Wf, and Wf is equal or smaller than Lf (Tf<Wf≤Lf). Lf, Wf and Tf can be further used to calculate an aspect ratio (e.g., Rf as below defined) of a matrix flake.

Such characteristic dimensions can be assessed on a number of representative particles, or a group of representative particles, that may accurately characterize the population (e.g., by diameter, longest dimension, thickness, aspect ratio and like characterizing measures of the particles). It will be appreciated that a more statistical approach may be desired for such assessments. When using microscopy for particle size characterization, a field of view of the image-capturing instrument (e.g., light microscope, etc.) is analyzed in its entirety. Typically, the magnification is adjusted such that at least 5 particles, at least 10 particles, at least 20 particles, or at least 50 particles are disposed within a single field of view. Naturally, the field of view should be a representative field of view as assessed by one skilled in the art of microscopic analysis. The average value characterizing such a group of particles in such a field of view is obtained by volume averaging. In such case, D_{V}50=Σ[(Deq(m))³/m]^{1/3}, wherein m represents the number of particles in the field of view and the summation is performed over all m particles. As mentioned, when such methods are the technique of choice for the scale of the particles to be studied or in view of their shape, such measurements can be referred to as D50.

As used herein, the terms “ultraviolet-protective agent” or “ultraviolet-protecting agent” refer to agents that absorb and/or reflect and/or scatter at least some of the UV radiation on surfaces exposed to sunlight or any other UV source, and thus reduce the effect of UV radiation on the surface. Typically, UV-protective agents provide at least 25% absorption of ultraviolet light in the wavelength range of from 290 nm to 400 nm, the exact range depending on whether the agents protect mainly from UVA radiation, UVB radiation or from both. The surface may be the skin and/or hair of a subject, such as a human subject. The surface may also be the surface (e.g., an exterior face) of an inanimate object.

In another aspect, embodiments of the present disclosure provide a method for the preparation of afore-described compositions.

Some known UV-protective compositions block both UVA and UVB radiation by use of a combination of different UV-protecting agents, each of which blocks radiation over a limited range of the UV spectrum.

As used herein, the term “broad-spectrum UV absorption” with regard to an ultraviolet-absorbing agent refers to an ultraviolet-absorbing agent that absorbs both UVA and UVB radiation. In some embodiments, the breadth of UV absorption may be measured according to the Critical Wavelength Method, wherein an ultraviolet-absorbing agent is considered to provide broad spectrum absorption when the critical wavelength is greater than 370 nm, and unless otherwise noted, in the present disclosure the term “broad-spectrum UV absorption” as used herein is determined on the basis of the critical wavelength.

As used herein, the term “critical wavelength” is defined as the wavelength at which the area under the absorbance spectrum from 290 nm to the critical wavelength constitutes 90% of the integral of the absorbance spectrum in the range from 290 nm to 400 nm.

In some instances, noted as such herein, the term “broad-spectrum UV absorption” with regard to an ultraviolet-absorbing agent refers to the situation in which the area under the curve (AUC) formed by the UV-absorption of the agent as a function of wavelength in the range of 280 nm to 400 nm (AUC₂₈₀₋₄₀₀) is at least 75% of the AUC formed by the same agent at the same concentration in the range of 280 nm to 700 nm (AUC₂₈₀₋₇₀₀). Similarly, where noted as such herein, the terms “broader-spectrum UV absorption” and “broadest spectrum UV absorption” with respect to a UV-absorbing agent refer respectively to the situation in which the area under the curve (AUC) formed by the absorption of the agent as a function of wavelength in the range of 280 nm to 400 nm (AUC₂₈₀₋₄₀₀) is at least 85% or 95% of the AUC formed by the same agent at the same concentration in the range of 280 nm to 700 nm (AUC₂₈₀₋₇₀₀).

As used herein, the term “ultraviolet-absorbing agent” refers to an agent which, when present in a composition at up to 50 wt. % of the total composition, provides at least 50% absorption of ultraviolet light in the wavelength range of from 290 nm to 400 nm. UV absorbing agents, in addition to the BLT crystals herein disclosed, can be organic or inorganic.

As used herein, the terms “substantially devoid of an organic ultraviolet-absorbing agent”, “essentially devoid of an organic ultraviolet-absorbing agent”, and “devoid of an organic ultraviolet-absorbing agent” refer respectively to a composition in which a UV-absorbing organic material, if any, is present in the composition at a concentration which provides absorption of not more than 20%, not more than 15%, not more than 10%, not more than 5%, not more than 2%, not more than 1% or not more than 0.5% of ultraviolet light in the wavelength range of from 290 nm to 400 nm.

As used herein, the term “substantially devoid of an additional ultraviolet-absorbing agent”, “essentially devoid of an additional ultraviolet-absorbing agent”, and “devoid of an additional ultraviolet-absorbing agent” refer respectively to a composition which is devoid of any UV-absorbing material other than that specifically disclosed as being present in the composition at a concentration, which, if included in the composition, provides absorption of not more than 20%, not more than 15%, not more than 10%, not more than 5%, not more than 2%, not more than 1% or not more than 0.5% of ultraviolet light in the wavelength range of from 290 nm to 400 nm.

According to an aspect of some embodiments, the present disclosure relates to compositions providing protection against ultraviolet radiation, and more particularly, to UV-protective compositions comprising a matrix comprising a polymer and a carrier (e.g., an oil-based of a water-based carrier), and doped or undoped BLT crystals and a dispersant, wherein the crystals or nanoparticles thereof are dispersed in the matrix. Advantageously, the dispersed crystals or nanoparticles thereof do not substantially migrate out of the polymer matrix. In such case, the crystals and nanoparticles thereof may also be said to be immobilised or embedded in the matrix, also referred to as the polymer matrix or the swelled polymer matrix.

According to an aspect of some embodiments of the disclosure, there is provided a matrix comprising a polymer and an oil-based or water-based carrier; and doped or undoped BLT crystals and a dispersant, dispersed in the matrix.

In some embodiments, the doped or undoped BLT crystals are present in the matrix at a concentration of from about 0.1 wt. % to about 60 wt. % of the polymer, or from about 3 wt. % to about 40 wt. %, optionally at a concentration of about 5 wt. % to 20 wt. % of the polymer of the matrix.

In some embodiments, the doped or undoped BLT crystals are present in the matrix at a concentration of from about 0.01 to about 8% (volume per volume or v/v) of the polymer, or from about 0.4 to about 5% (v/v), optionally at a concentration of about 0.6 to about 3% (v/v) of the polymer of the matrix.

In some embodiments, the doped or undoped BLT crystals are present in the matrix at a concentration of from about 1 to about 10% (weight per weight, w/w or wt. %) or from about 1 to about 10% (v/v) of the total composition, optionally at a concentration of about 4% (w/w) or 0.5% (v/v) of the composition.

In some embodiments, the oil-based or water-based carrier is present in the polymer matrix at a concentration of from about 10 to about 50% (w/w) of the polymer of the matrix or from about 5 to about 50% (v/v) of the polymer of the matrix, optionally at a concentration of about 30% (w/w) or about 20% (v/v) of the polymer of the matrix.

In some embodiments, the oil-based carrier of the UV-protective composition and/or of the matrix is selected from the group consisting of mineral oil, natural oil, vegetal oil, synthetic oil, and combinations thereof. In a particular embodiment, the oil is a C₁₀₋₁₅ hydrocarbon such as isoparaffin or C₁₂-C₁₅ alkyl benzoate.

In some embodiments, the water-based carrier of the UV-protective composition and/or of the matrix is selected from the group consisting of water, glycols having a molecular weight of up to 800 gr/mole, C₁₋₅ alcohols and glycerol.

In some embodiments, the polymer of the matrix is a swellable thermoplastic homo- or co-polymer, optionally clear, transparent and/or colorless. In particular embodiments, the thermoplastic polymer of the matrix is swellable by the oil-based carrier.

The carriers optionally used in the UV-protective composition and in the polymer matrix, when at least part of the BLT nanoparticles are embedded therein, are typically of the same type, but need not be identical, as long as compatible. For instance, the UV-protective composition may contain a first oil-based carrier and nanoparticles of doped or undoped BLT crystals dispersed within a polymer matrix swelled with a second oil-based carrier, the first and second oil-based carrier being either the same or different.

In some preferred embodiments, the polymers suitable for the matrix are functionalized polymers or copolymers comprising particle-affinic functional group and non-affinic monomer units. For instance, the functional groups may be acidic monomers, whereas the non-affinic groups can be ethylene. In some embodiments, the polymer comprises at least one ethylene-acrylic (EAA) polymer, ethylene-methacrylic (EMMA) polymer, ethyl vinyl acetate (EVA) polymer, and combinations thereof.

In some embodiments, the polymer of the matrix comprises at least one ethylene-acrylic polymer, optionally wherein the ethylene-acrylic polymer comprises from about 5 wt. % to about 30 wt. % acrylic monomer. In some embodiments, the ethylene-acrylic polymer is selected from the group consisting of ethylene-methacrylic acid copolymer and ethylene-acrylic acid copolymer.

In some embodiments, the polymer of the matrix, which can be a copolymer or a combination thereof, have at least one of a softening point and a melting point not exceeding 200° C., said softening point or melting point optionally being of at least 60° C.

The oil-based or water-based carrier and the polymer of the polymer matrix, or a combination of carriers and/or a combination of polymers forming such a matrix, are selected and adapted to be compatible one with the other. In other words, the carrier(s) can swell the polymer(s) and the polymer(s) can be swelled by the carrier(s). While being swellable by the carrier, the polymer does not dissolve within it, i.e. less than about 5% by weight of the polymer dissolves within the carrier. Swelling (and grammatical variants) refers to the ability of the carrier to penetrate a polymeric network formed by the polymer (the matrix), causing a decrease in the attraction of the polymeric chains, and resulting, among other things, in an increase in the weight of the matrix, and typically additionally in an expansion of its volume. Swelling of the polymer within its carrier typically decreases the polymer viscosity, rendering it more malleable.

In some embodiments, the dispersant adapted to disperse the nanoparticles of doped or undoped BLT crystals within the polymeric matrix and the carrier used for polymer-swelling are compatible with one another, such that at least 80% of the dispersant dissolves within the carrier. Thus, depending on the type of compatible carrier, the dispersant can be either termed an oil-based dispersant or a water-based dispersant.

In some embodiments, the oil-based dispersant has a hydrophilic-lipophilic balance (HLB) value of at most 9, at most 6, at most 4, or at most 3. In some embodiments, the water-based dispersant has an HLB value of at least 9, at least 10, at least 11 or at least 12.

While the dispersants used in the manufacturing of the nanoparticles and in their later dispersion in a polymeric matrix, if present, need preferably be compatible, they need not be identical. Likewise for the carriers that may be used in different steps leading to the preparation of the final UV-protective composition, while chemical similarity can be preferred to increase compatibility, any miscible carriers can be used, and they need not be identical.

In some embodiments, the dispersant used for dispersing the nanoparticles of BLT within the polymer matrix is oleic acid, polyhydroxystearic acid (such as commercially available from Innospec Performance Chemicals, USA, under tradenames Dispersun DSP-OL100 and DSP-OL300, or from Phoenix Chemicals, USA, under the tradename Pelemol® PHS-8) or octyldodecyl/PPG-3 myristyl ether dimer dilinoleate (such as commercially available as PolyEFA from Croda Inc., UK).

Non-limiting examples of dispersants suitable for the preparation of the nanoparticles and/or the dispersion of the nanoparticles within the polymer matrix include: polyacrylic acid and salts thereof, e.g. sodium salt (PAA, such as commercially available from Sigma Aldrich, USA, under the CAS nos. 9003-01-4, for the acid form, and 9003-04-7, for the sodium salt form), a polyhydroxystearic acid, oleic acid, octyldodecyl/PPG-3 myristyl ether dimer dilinoleate, and any of the Pelemol esters, available commercially from Phoenix Chemicals, USA: Pelemol® BIP-PC (butylphthalimide combined with isoproplylphthalimide); Pelemol® C25EH (C₁₂₋₁₅ alkyl ethylhexanoate); cetyl esters such as Pelemol® CA (cetyl acetate) and Pelemol® 168 (cetyl ethylhexanoate); Pelemol® 899 (isononyl isononanoate combined with ethylhexyl isonononoate); Pelemol® 256 (C₁₂₋₁₅ alkyl benzoate); Pelemol® 89 (ethylhexyl isononanoate); Pelemol® 3G22 (polyglyceryl-3 behenate); Pelemol® D5R1 (ethyl isonanoate combined with cetyl dimethicone); Pelemol® D5RV (propanediol dicaprylate/caprate combined with diisostearyl malate); Pelemol® D899 (PPG-26 dimer dilinoleate copolymer combined with isononyl isononanoate and with ethylhexyl isononanoate); Pelemol® DD (dimer dilinoleyl dimer dilinoleate); Pelemol® DDA (diethylhexyl adipate); Pelemol® DO (decyl oleate); Pelemol® DP-72 (dipentaerythrityl tetrahydroxystearate/tetraisostearate); Pelemol® EE (octyldodecyl erucate); glyceryl esters such as Pelemol® G7A (glyceryl-7 triacetate), Pelemol® GMB (glyceryl behemate), Pelemol® GMR (glyceryl ricinoleate) and Pelemol® GTAR (glyceryl triacetyl ricinoleate); Pelemol® GTB (tribehenin); Pelemol® GTHS (trihydroxystearin); Pelemol® GTIS (triisostearin); Pelemol® GTO (triethylhexanoin); Pelemol® ICB (isocetyl behenate); Pelemol® IN-2 (isononyl isonanoate), isostearyl esters such as Pelemol® II (isostearyl isostearate), Pelemol® ISB (isostearyl behenate), Pelemol® ISHS (isostearyl hydroxystearate) and Pelemol® ISNP (isostearyl neopentanoate); Pelemol® JEC (triisostearin/glyceryl behenate); Pelemol® MAR (methyl acetyl ricinoleate); Pelemol® NPGDD (neopentylglycol dicaprate/dicaprylate); Pelemol® OL (oleyl lactate); Pelemol® OPG (ethylhexyl pelargonate); Pelemol® P-49 (pentaerylthrityl tetraisononanoate); Pelemol® P-810 (propanediol dicaprylate/caprate); Pelemol® P-1263 (polyglycerol-10 hexaoleate combined with polyglyceryl-6 poyricinoleate); pentaerythrityl esters such as Pelemol® PTIS (pentaerythrityl tetraisostearate), Pelemol® PTL (pentaerythrityl tetralaurate) and Pelemol® PTO (pentaerythrityl tetraethylhexanoate); Pelemol® SPO (cetearyl ethylhexanoate); Pelemol® TDE (tridecyl enucate); Pelemol® TGC (trioctyldodecyl citrate); trimethylolpropane esters such as Pelemol® TMPIS (trimethylolpropane triisostearate) and Pelemol® TMPO (Trimethylolpropane Triethylhexanoate); Pelemol® TT (tribeherin combined with caprylic/capric triglyceride); and Pelemol® VL (dimer dilinoelyl dimer dilinoleate combined with triisostearin).

In some embodiments, the matrix is present in the form of matrix elements, at least 50% of the number of matrix elements having at least one dimension of up to about 50 μm, at most 25 μm, at most 10 μm or at most 5 μm.

In some embodiments, the matrix elements of the polymer matrix (e.g., comprising a thermoplastic polymer swelled with an oil and nanoparticles of doped or undoped BLT crystals dispersed and embedded therein with a dispersant) are matrix flakes, wherein each flake of the swelled polymer matrix flakes has a flake length (Lf), a flake width (Wf), and a flake thickness (Tf), the matrix flakes having a dimensionless flake aspect ratio (Rf) defined by:

Rf=(Lf·Wf)/(Tf)²

wherein, with respect to a representative group of the swelled polymer matrix flakes, an average Rf is at least 5.

In some embodiments, at least one of the flake length (Lf) and the flake width (Wf) of the matrix flakes is at most 50 μm, at most 25 μm, at most 10 μm, or at most 5 μm.

In some embodiments, the flake thickness (Tf) of the matrix flakes is at most 1000 nm, at most 900 nm, at most 750 nm, at most 650 nm, at most 600 nm, at most 550 nm, at most 500 nm, at most 450 nm, at most 400 nm, at most 350 nm, at most 300 nm, or at most 250 nm.

In some embodiments, flake aspect ratio (Rf) of the matrix flakes is within a range of from about 5 to about 2000, from about 10 to about 1000, from about 12 to about 500, from about 12 to about 200, or from about 15 to about 100.

In some embodiments, the representative group is disposed in an instrumental field of view containing at least 10 of the matrix flakes or swelled polymer matrix flakes, and optionally hundreds of nanoparticles of doped or undoped BLT crystals.

In some embodiments, at least 50%, at least 60%, at least 75%, or at least 90% of the nanoparticles embedded in the matrix elements or matrix flakes have a cumulative particle size (D50, D60, D75, and D90, accordingly) of at most 100 nm, at most 90 nm, at most 80 nm, at most 70 nm, or at most 60 nm. The cumulative particle size can be determined in terms of percent number of nanoparticles in the population of the plurality of particles or in terms of percent volume. Thus, in some embodiments, the nanoparticles of BLT crystals embedded in the matrix flakes can be characterized by a D_{N}50 of at most 100 nm (up to a D_{N}90 of at most 60 nm) or by a D_{V}50 of at most 100 nm (up to a D_{V}90 of at most 60 nm).

According to an aspect of some embodiments, the present disclosure relates to a method of preparing UV-protective compositions comprising doped or undoped BLT nanoparticles dispersed in a matrix comprising a polymer, a carrier (an oil-based or water-based carrier) and a dispersant. The method comprises embedding the nanoparticles within the polymeric matrix, the polymer being swelled by the carrier.

Nanoparticles are not easily dispersed or embedded within a polymer matrix, due to unfavorable entropic interactions between the matrix and the nanoparticles. Thus, simply mixing together the nanoparticles with the polymers would usually result in the nanoparticles being disposed on the polymer surface, rather than being embedded within the matrix. In view of this challenge, methods for preparing thermodynamically stable polymeric dispersions of nanoparticles would require some effort.

One such method involves core-shell techniques, wherein monomers are adsorbed onto the surface of the particle, and subsequently undergo polymerization. The resulting particles (usually spherical) are formed, bottom-up, having the nanoparticle placed in their “core”, encapsulated by the polymeric “shell”. Fluidized bed methodology is another conventional option, wherein nanoparticles are suspended in polymers liquidized within a solvent, following by evaporation of the solvent, resulting in the nanoparticles being coated with the polymer.

The method of the present invention, in comparison and contrast, encompasses milling the nanoparticles together with the polymeric matrix, including the dispersant and oil-based or water-based carrier, which allows for embedding of the nanoparticles into the solid polymeric matrix. The present method thus typically allows the inclusion of a plurality of nanoparticles of BLT within the polymeric matrix elements, the nanoparticles being well dispersed therein as individual discrete particles.

In some embodiments, the UV-protective composition provides protection against UV radiation selected from the group consisting of a UVA-radiation and a UVB-radiation. In some embodiments, the UV-protective composition provides UVA- and UVB-protective activity. In some embodiments, when using the UV-protective compositions for inanimate objects, the compositions may comprise nanoparticles that provide protection mostly against UVB radiation.

Aspects and embodiments of the disclosure are described in the specification herein below and in the appended claims.

Unless otherwise defined, all technical and scientific terms used herein have the same meaning as commonly understood by one of ordinary skill in the art to which the particular teachings pertain. In case of conflict, the specification, including definitions, will take precedence.

As used herein, the terms “comprising”, “including”, “having” and grammatical variants thereof are to be taken as specifying the stated features, integers, steps or components, but do not preclude the addition of one or more additional features, integers, steps, components or groups thereof.

As used herein, the indefinite articles “a” and “an” and the singular form “the” include plural references and mean “at least one” or “one or more” unless the context clearly dictates otherwise. At least one of A and B is intended to mean either A or B, and may mean, in some embodiments, A and B.

Unless otherwise stated, the use of the expression “and/or” between the last two members of a list of options for selection indicates that a selection of one or more of the listed options is appropriate and may be made.

In the discussion, unless otherwise stated, adjectives such as “substantially” and “about” that modify a condition or relationship characteristic of a feature or features of an embodiment of the present technology, are to be understood to mean that the condition or characteristic is defined within tolerances that are acceptable for operation of the embodiment for an application for which it is intended, or within variations expected from the measurement being performed and/or from the measuring instrument being used. In particular, when a numerical value is preceded by the term “about”, the term “about” is intended to indicate +/−15%, or +/−10%, or +/−5%, or +/−2% of the mentioned value and in some instances the precise value.

Additional objects, features and advantages of the present teachings, and aspects of embodiments of the invention, will be set forth in the detailed description which follows, and in part will be readily apparent to those skilled in the art from the description or recognized by practicing embodiments of the invention as described in the written description and claims hereof, as well as the appended drawings. Various features and sub-combinations of embodiments of the present disclosure may be employed without reference to other features and sub-combinations.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

It is to be understood that both the foregoing general description and the following detailed description, including the materials, methods and examples, are merely exemplary, and are intended to provide an overview or framework to understanding the nature and character of the invention as it is claimed, and are not intended to be necessarily limiting. Many other alternatives, modifications and variations of such embodiments will occur to those skilled in the art based upon Applicant's disclosure herein. Accordingly, it is intended to embrace all such alternatives, modifications and variations and to be bound only by the spirit and scope of the disclosure and any change which come within their meaning and range of equivalency.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

To the extent necessary to understand or complete the disclosure of the present disclosure, all publications, patents, and patent applications mentioned herein, including in particular the applications of the Applicant, are expressly incorporated by reference in their entirety by reference as is fully set forth herein.

Certain marks referenced herein may be common law or registered trademarks of third parties. Use of these marks is by way of example and shall not be construed as descriptive or limit the scope of this disclosure to material associated only with such marks.

### BRIEF DESCRIPTION OF THE DRAWINGS

Some embodiments of the invention are described herein with reference to the accompanying figures. The description, together with the figures, makes apparent to a person having ordinary skill in the art how some embodiments of the disclosure may be practiced. The figures are for the purpose of illustrative discussion and no attempt is made to show structural details of an embodiment in more detail than is necessary for a fundamental understanding of the disclosure. For the sake of clarity, some objects depicted in the figures are not to scale.

In the Figures:

FIG. 1 is a plot showing the powder X ray diffraction (PXRD) diffractogram of Fe-doped and undoped BLT crystals prepared according to the present teachings.

FIG. 2 is a line graph showing powder absorbance of Fe-doped and undoped BLT crystals prepared according to present teachings, as compared to the mixtures of their respective constituents, BLTO-Fe and BLTO.

FIG. 3 is a line graph showing powder absorbance of BLT crystals doped with various ratios of iron to titanium atoms as prepared according to present teachings, as compared to undoped BLT crystals as reference.

FIG. 4 is a line graph showing Particle Size Distribution (PSD) of particles of Fe-doped and undoped BLT crystals in aqueous dispersions after milling according to present teachings, expressed as number percentage.

FIG. 5 is a line graph showing absorbance of aqueous suspensions comprising different concentrations of nanoparticles of undoped BLT crystals prepared according to present teachings, as compared to a commercial sample and a control consisting of nanoparticles of Zinc Oxide.

FIG. 6 is a line graph showing absorbance of aqueous suspensions comprising same concentration of nanoparticles of BLT crystals at various levels of Fe-doping prepared according to present teachings, as compared to undoped BLT.

FIGS. 7A-7B are Scanning Transmitting Electron Microscopy (STEM) images captured using a high-resolution Scanning Electron Microscope (HR-SEM) of nanoparticles of BLT crystals prepared according to present teachings, where FIG. 7A shows nanoparticles of undoped BLT and FIG. 7B shows nanoparticles of Fe-doped BLT. The scale bar in the pictures represent 100 nm.

FIG. 8 is a line graph showing Particle Size Distribution of particles of Fe-doped BLT crystals (Fe:Ti 1:2 and Fe:Ti 0.25:2.75) in non-aqueous dispersions after milling according to present teachings, expressed as number percentage.

FIG. 9 is a STEM image captured using a HR-SEM microscope of nanoparticles of Fe-doped BLT (Fe:Ti 1:2) crystals prepared according to present teachings, dispersed in a non-aqueous dispersion. The scale bar in the picture represents 20 nm.

FIG. 10 is a STEM image captured using a HR-SEM microscope of nanoparticles of Fe-doped BLT (Fe:Ti 0.25:2.75) crystals prepared according to present teachings, dispersed in a non-aqueous dispersion. The scale bar in the picture represents 100 nm.

FIG. 11 is a line graph showing Particle Size Distribution of swelled polymer matrix macroparticles containing nanoparticles of Fe-doped BLT (Fe:Ti 1:2 and Fe:Ti 0.25:2.75) prepared according to the present teachings, expressed as volume percentage.

FIG. 12 is a STEM image captured using a HR-SEM microscope of swelled polymer matrix macroparticles including Fe-doped BLT (Fe:Ti 0.25:2.75) crystals prepared according to present teachings. The scale bar in the picture represents 200 nm.

FIG. 13 is a line graph showing absorbance of non-aqueous dispersions comprising swelled polymer matrix macroparticles incorporating Fe-doped BLT (Fe:Ti 1:2 and Fe:Ti 0.25:2.75) nanoparticles according to the present teachings.

### DETAILED DESCRIPTION

The present disclosure, in at least some embodiments, provides compositions for protection against ultraviolet radiation, uses of such compositions and methods of making such compositions.

The UV-protective compositions disclosed herein comprise Fe-doped or undoped BLT crystals having the formula Bi₍₄₋ₓ₎La₍ₓ₎Ti_{(3-y)}Fe_{(y)}O₁₂, wherein x is between 0.1 and 1.5; and wherein y is between 0 and 2, which when present as large particles (e.g., dimensions in each of the X-, Y- and Z-directions being greater than 250 nanometers (nm), resulting for instance in a hydrodynamic diameter of more than 500 nm as measured by DLS) may effectively absorb radiation having wavelengths of greater than about 400 nm. Accordingly, compositions comprising such large particles of composite BLT, whether or not further substituted by iron atoms, may provide protection against ultraviolet radiation having wavelengths up to at least 400 nm. There may be instances where particles having a hydrodynamic diameter of more than 250 nm (but not more than 500 nm) are used as well, e.g. in the preparation of coatings for inanimate objects, wherein some degree of tinting is tolerable or even required, or for cosmetic compositions wherein tinting might be desirable.

However, in the case in which the UV-protective composition is a sunscreen composition which comprises BLT, but which also contains particles that absorb light at wavelengths in the range of 400-800 nm, the sunscreen will be visible on the end-user because of the absorption in the visible range (>400 nm).

It has surprisingly been found by the present Inventors that, although reduction of particle size of known inorganic UV-absorbing agents to dimensions of, for example below 1 micrometer (μm), typically below 100 nm (for instance, reduction to nanometric dimensions) is known to significantly reduce the maximum wavelength of light, including UV light, which is effectively absorbed by the particles, UV-protective compositions according to the present teachings comprising particles of doped or undoped BLT crystals milled to nanoparticle size still provide substantial absorption of UV radiation of wavelength from 280 nm (or even shorter wavelength) up to about 400 nm, thus providing broad-spectrum protection against both UVA and UVB radiation, even in the absence of additional ultraviolet-absorbing agents.

Thus, in some embodiments, UV-protective compositions disclosed herein, such as sunscreen compositions, comprise doped or undoped BLT crystals in the form of particles comprising one or more said crystals, wherein at least 90% of the particles are nanoparticles. In some embodiments, at least 95%, or at least 97.5% or at least 99% of the particles, in terms of number or volume of particles, are nanoparticles. In some embodiments, at least one dimension of the BLT crystal nanoparticles is expressed in terms of the hydrodynamic diameter as measured by DLS techniques.

In some embodiments, the cumulative particle size distribution in a sample is assessed in terms of the number of particles in the sample (denoted D_{N}). In some embodiments, the cumulative particle size distribution in a sample is assessed in terms of the volume of particles in the sample (denoted D_{V}).

In some embodiments, the maximum diameter of the nanoparticles is assessed for population distribution measured in terms of number of particles and percentage thereof. In some embodiments, the maximum diameter of the nanoparticles is assessed for population distribution measured in terms of sample volume of particles and percentage thereof.

In some embodiments, the doped or undoped BLT crystal nanoparticles are substantially invisible to the human eye, in particular when applied to the skin or hair of a subject, or if desired when applied to an inanimate surface, due to their small size.

In some embodiments, the doped or undoped BLT crystal nanoparticles are blended into a colored composition and need not be substantially transparent and/or invisible, for instance when used in a make-up product, such as a foundation, which is slightly tinted when applied to the skin of a subject, or when used in a stain or paint applicable to inanimate surfaces.

According to some embodiments of the disclosure, there is provided a UV-protective composition comprising undoped BLT crystals.

According to some embodiments of the disclosure, there is provided a UV-protective composition comprising Fe-doped BLT crystals, the level of doping by iron atoms being such that the Fe:Ti ratio can be between 1:299 and 1:2, between 1:200 and 1:2, between 1:100 and 1:2 or between 1:50 and 1:2. In particular embodiments, the Fe:Ti ratio can be between 1:20 and 1:2.

According to a further aspect of some embodiments of the disclosure, there is provided a method of preparing doped or undoped BLT nanoparticles from powders of metal oxides, metal nitrates or metal carbonates. The method comprises combining powders of lanthanum, bismuth and titanium, each in the form of either oxides, nitrates or carbonates, in appropriate ratios so as to obtain the desired stochiometric amount. In particular embodiments, lanthanum oxide (La₂O₃), bismuth oxide (Bi₂O₃) and titanium dioxide (TiO₂) are combined.

For preparing Fe-doped BLT, some of the metal starting material including titanium is replaced with a starting material including iron. The extent of the replacement is determined according to the desired Fe-doping level, wherein a lower doping level of 0.5 or less results in a narrower UV-spectrum protection (which can be used for inanimate objects), and a higher doping level of above 0.5 results in a broader UV-spectrum protection, more beneficial for cosmetic use. The amount of the added ferric oxide (particularly Fe₂O₃) is calculated to provide the intended Fe-doping ratio, and a corresponding amount of the titanium starting material is reduced accordingly.

The powdered metal starting materials are then mixed until homogenization by any means known in the art (e.g., by a mortar grinder). As used herein, the term “homogenous” (and grammatical variants), refer to a mixture, which components are uniformly distributed throughout, forming a single phase.

Following homogenization, the mixture is calcinated, under conditions which can be readily determined by anyone skilled in the art without undue experimentation. In a particular embodiment, when metal oxides are used as starting materials, calcination is conducted at about 1000° C. for approximately 24 hours. Calcination is performed in order to form crystals of the Fe-doped or undoped BLT substance from the individual powders of metal starting materials, while removing any volatile substance in the process.

Following calcination, the obtained doped or undoped BLT crystals are then allowed to cool down to ambient temperature (circa 23° C.), followed by low-energy milling (e.g. by a mortar grinder or ball mill). Low-energy milling suffices to break down the calcinated material into smaller chunks of a size suitable for the following steps.

For the nanoparticles preparation, the low-energy milled particles are combined with a dispersant, optionally in the presence of an oil-based or water-based carrier, and the obtained slurry is then high-energy milled, whereby nanoparticles of doped or undoped BLT are obtained.

The types of dispersant and optional oil-based or water-based carrier that can be used in the high-energy milling step depend on the further processing of the nanoparticles, as well as the intended use of the compositions containing them.

So, for example, if the intended UV-protective composition is a sunscreen composition to be applied on the skin, such a composition might preferably be prepared using oil-based constituents, to provide water-resistance (e.g., to sweat or swimming environment). In such a case, it might be further preferred to disperse the nanoparticles of BLT in a polymer matrix, and having such an illustrative purpose in mind, it would be advantageous to use an oil-based dispersant and optionally an oil-based carrier for the manufacturing of the nanoparticles, as well as an oil-based dispersant and oil-based swelling liquid or carrier for the polymeric matrix. The obtained mixture is compatible in the sense that no turbidity or phase separation is observed in the final UV-protective composition.

Suitable equipment for the nanoparticles grinding or high-energy milling may include an attritor media grinding mill, a high-energy ball mill, a dyno mill, a zeta mill and a sonicator to name a few.

While nanoparticles can in theory be prepared by various methods, only a few might be appropriate for industrial manufacturing of significant amounts of composite materials within a reasonably short time period. Bottom-up methods, e.g. growing the crystals in highly diluted solutions, may be inadequate for large scale production. Top-down methods may provide relatively more concentrated compositions than the former method, the composite material being at the end of the process for its preparation generally milled by low-energy milling methods (such as ball milling). Low-energy milling methods are typically capable of breaking-down chunks of materials into smaller macro-particles in the size range of millimetres to micrometres depending on the duration of the milling. While smaller particles could eventually be produced by low-energy milling, such sub-micron particles would typically not exceed 10% of the entire population of the particles so produced. Thus, top-down methods typically result in the formation of agglomerates and/or impure composites, depending on the preparation method. Agglomerates having at least one dimension even in the range of micrometers, will scatter incident light and will therefore be inappropriate for the preparation of transparent compositions according to aspects of the present teachings.

In contrast, the method of the present invention encompasses a top-down preparation of doped or non-doped BLT nanoparticles, whereby the mixed powders are calcinated to obtain a bulk of agglomerated crystals, which is later ground by high-energy milling in the presence of a compatible dispersant, allowing to obtain discrete, individual nanoparticles. High-energy milling, in contrast with previously described low-energy method, allows for the preparation of particles predominantly in the sub-micron range, advantageously in the range of no more than 500 nm, no more than 250 nm, no more than 200 nm or no more than 100 nm. While particles milled by a high-energy milling method (e.g., a sonicator), may include some particles in the range of a few micrometers, such methods are typically employed for a duration of time or at an efficiency such that the larger particles in the micron range do not exceed 10% of the entire population of particles.

Without wishing to be bound by theory, the inventors believe that for particles that absorb light in the UV-Visible range, downsizing the particles to the nanometric scale may effect a “blue shift” in the absorbance band, often on the order of 100 to 200 nm.

This phenomenon occurs when the downsizing produces discrete nanoparticles. For nanopowders that are not dispersed in a medium, however, the absorption profile may be substantially similar to that of the bulk material. Moreover, the inventors believe that in some cases, the size reduction process may introduce enough stress, strain, or defects into the nano-crystalline structures such that the absorption profile may be deleteriously affected. In severe cases, the obtained material may actually become useless as a UV-absorbing agent.

According to a further aspect of some embodiments of the disclosure, there is provided a UV-protective composition comprising doped or undoped BLT crystals for use in protecting the skin of a subject, such as a human subject, against ultraviolet radiation, in some embodiments providing broad-spectrum protection against both ultraviolet A and ultraviolet B radiation.

According to a further aspect of some embodiments of the disclosure, there is provided a UV-protective composition comprising doped or undoped BLT crystals for use in protecting the hair of a subject, such as a human subject, against ultraviolet radiation, in some embodiments against both ultraviolet A and ultraviolet B radiation.

According to a further aspect of some embodiments of the disclosure, there is provided a UV-protective composition comprising doped or undoped BLT crystals for use in protecting the surface of an inanimate object against ultraviolet radiation, in some embodiments against both ultraviolet A and ultraviolet B radiation and in other embodiments mainly against ultraviolet B radiation.

According to a further aspect of some embodiments of the disclosure, there is provided a method of protecting the skin of a subject against ultraviolet radiation, the method comprising applying to the skin of the subject an efficacious amount of a UV-protective composition comprising doped or undoped BLT crystals. In some such embodiments, the UV-protective composition can be in the form of a skin-care product suitable for skin application and/or at least temporary retention thereupon.

According to a further aspect of some embodiments of the disclosure, there is provided a method of protecting the hair of a subject against ultraviolet radiation, the method comprising applying to the hair of the subject an efficacious amount of a UV-protective composition comprising doped or undoped BLT crystals. In some such embodiments, the UV-protective composition can be in the form of a hair-care product suitable for hair application and/or at least temporary retention thereupon.

According to a further aspect of some embodiments of the disclosure, there is provided a method of protecting the surface of an inanimate object against ultraviolet radiation, the method comprising applying to the surface of the object an efficacious amount of a UV-protective composition comprising doped or undoped BLT crystals. In some such embodiments, the UV-protective composition can be in the form of a coating product suitable for application to inanimate surfaces and/or at least temporary retention thereupon.

According to a further aspect of some embodiments of the disclosure, there is provided the use of doped or undoped BLT crystals in the manufacture of a composition for protection of the skin of a subject against ultraviolet radiation.

According to a further aspect of some embodiments of the disclosure, there is provided the use of doped or undoped BLT crystals in the manufacture of a composition for protection of the hair of a subject against ultraviolet radiation.

According to a further aspect of some embodiments of the disclosure, there is provided the use of doped or undoped BLT crystals in the manufacture of a composition for protection of surfaces of an object against ultraviolet radiation.

According to a further aspect of some embodiments of the disclosure, there is provided a method of manufacturing a UV-protective composition, comprising combining doped or undoped BLT crystals, as an ultraviolet-absorbing agent, with other ingredients in proportions and in a manner suitable to make a UV-protective composition as described herein.

In some embodiments of the composition, use or method disclosed herein, the BLT crystals are present in the composition at a concentration of from about 0.001 wt. % to about 40 wt. %, from about 0.01 wt. % to about 30 wt. %, from about 0.1 wt. % to about 20 wt. % or from about 0.1 wt. % to about 15 wt. % of the final composition.

In some embodiments, the BLT crystals constitute at least 0.01 wt. %, at least 0.1 wt. %, at least 0.5 wt. %, at least 1 wt. %, at least 2 wt. %, at least 3 wt. %, at least 4 wt. %, at least 5 wt. %, at least 10 wt. %, at least 15 wt. %, at least 20 wt. %, at least 25 wt. %, at least 30 wt. %, or at least 35 wt. % of the composition. In some embodiments, the BLT crystals constitute at most 40 wt. %, at most 35 wt. %, at most 30 wt. %, at most 25 wt. %, at most 20 wt. %, at most 15 wt. %, at most 10 wt. %, at most 5 wt. %, at most 4 wt. %, at most 3 wt. %, at most 2 wt. %, at most 1 wt. %, at most 0.5 wt. %, or at most 0.1 wt. % of the composition.

In some embodiments of the composition, use or method disclosed herein, the doped or undoped BLT crystals are present in the composition as nanoparticles having at least one dimension of up to about 500 nm. In some embodiments, the nanoparticles have at least one dimension in the range of from about 10 nm to about 500 nm, from about 20 nm to about 500 nm, from about 10 nm to about 400 nm, from about 10 nm to about 300 nm, from about 10 nm to about 250 nm, from about 10 nm to about 200 nm, from about 20 nm to about 150 nm, from about 20 to about 100 nm, from about 10 nm to about 80 nm, from about 10 to about 70 nm, from about 20 to about 70 nm, or from about 20 to about 60 nm, In some particular embodiments, the nanoparticles have at least one dimension of about 30 nm.

In some embodiments, the afore-mentioned dimensions or ranges of dimensions apply to at least 95%, or at least 97.5% or at least 99% of the population of the nanoparticles.

In some embodiments, the aforesaid smallest dimension of doped or undoped BLT crystals is estimated based on the hydrodynamic diameter of the particles as measured by DLS techniques. In some embodiments, the population distribution of the particles is expressed in terms of the cumulative particle size distribution, according to the number of particles in a sample. In some embodiments, the population distribution of the particles is expressed in terms of the cumulative particle size distribution of a sample volume of particles.

In some embodiments of the composition, use or method disclosed herein, the composition is generally devoid and/or generally free of an organic ultraviolet-absorbing agent.

In some embodiments of the composition, use or method disclosed herein, the composition is generally free of an organic ultraviolet-absorbing agent, that is to say the composition contains less than 5 wt. % organic UV-absorbing agents. In some embodiments the composition contains less than 4 wt. %, less than 3 wt. %, less than 2 wt. % or less than 1 wt. % organic UV-absorbing agents. In some embodiments the composition is largely free of organic ultraviolet-absorbing agents, i.e. the composition contains less than 0.5 wt. % organic UV-absorbing agents. In some embodiments the composition is mostly free of organic UV-absorbing agents, i.e. the composition contains less than 0.1 wt. % organic UV-absorbing agents. In some embodiments, the composition is principally free of organic ultraviolet-absorbing agents, i.e. the composition contains less than 0.05 wt. % organic UV-absorbing agents. In some embodiments, the composition is fundamentally free of organic UV-absorbing agents, i.e. the composition contains less than 0.01 wt. % organic UV absorbing agents. In some embodiments of the composition, use or method disclosed herein, the composition is generally devoid of organic ultraviolet-absorbing agents, considerably devoid of organic ultraviolet-absorbing agents, significantly devoid of organic ultraviolet-absorbing agents, substantially devoid of organic ultraviolet-absorbing agents, essentially devoid of organic ultraviolet-absorbing agents, substantively devoid of organic ultraviolet-absorbing agents or devoid of organic ultraviolet-absorbing agents.

In some embodiments of the composition, use or method disclosed herein, the composition is generally devoid and/or generally free of an additional inorganic ultraviolet-absorbing agent.

In some embodiments of the composition, use or method disclosed herein, the composition is generally free of an additional inorganic ultraviolet-absorbing agent, that is to say the composition contains less than 5 wt. % additional inorganic UV-absorbing agents. In some embodiments, the composition contains less than 4 wt. %, less than 3 wt. %, less than 2 wt. % or less than 1 wt. % additional inorganic UV-absorbing agents. In some embodiments, the composition is largely free of additional inorganic ultraviolet-absorbing agents, i.e. the composition contains less than 0.5 wt. % additional inorganic UV-absorbing agents. In some embodiments, the composition is mostly free of additional inorganic UV-absorbing agents, i.e. the composition contains less than 0.1 wt. % additional UV-absorbing agents. In some embodiments, the composition is principally free of additional inorganic ultraviolet-absorbing agents, i.e. the composition contains less than 0.05 wt. % additional UV-absorbing agents. In some embodiments, the composition is fundamentally free of additional inorganic UV-absorbing agents, i.e. the composition contains less than 0.01 wt. % additional UV absorbing agents.

In some embodiments of the composition, use or method disclosed herein, the composition is generally devoid of additional ultraviolet-absorbing agents, considerably devoid of additional ultraviolet-absorbing agents, significantly devoid of additional ultraviolet-absorbing agents, substantially devoid of additional ultraviolet-absorbing agents, essentially additional of organic ultraviolet-absorbing agents, substantively devoid of additional ultraviolet-absorbing agents or devoid of additional ultraviolet-absorbing agents.

In some embodiments of the composition, use or method disclosed herein, the doped or undoped BLT crystals are the sole ultraviolet-absorbing agent.

In some embodiments of the composition, use or method disclosed herein, the composition further comprises silver metal particles.

In some embodiments, the silver metal particles are present in the composition as nanoparticles. In some embodiments, the silver nanoparticles have at least one dimension of up to about 50 nm. In some embodiments, the silver nanoparticles have at least one dimension of up to about 40 nm. In some embodiments, the silver nanoparticles have at least one dimension of up to about 30 nm. In some embodiments, the silver nanoparticles have at least one dimension in the range of from about 10 nm to up to about 50 nm.

In some embodiments, the afore-mentioned dimensions or ranges of dimensions apply to at least 90%, or at least 95%, or at least 97.5% or at least 99% of the population of the silver nanoparticles.

In some embodiments, the aforesaid at least one dimension of the silver nanoparticles is estimated based on the hydrodynamic diameter of the particles as measured by DLS techniques. In some embodiments, the population distribution of the particles is expressed in terms of the cumulative particle size distribution according to the number of particles in a sample. In some embodiments, the population distribution of the particles is expressed in terms of the cumulative particle size distribution of a sample volume of particles.

In some embodiments, the silver nanoparticles are present in the composition at a concentration in the range of from about 0.01 wt. % to about 10 wt. % of the total composition. In some embodiments, the silver nanoparticles are present in the composition at a concentration in the range of from about 0.01 wt. % to about 5 wt. %, from about 0.05 wt. % to about 5 wt. %, or from about 0.1 wt. % to about 2 wt. % of the total composition. In some preferred embodiments, the silver nanoparticles are present in the composition at a concentration of about 1 wt. % or about 2 wt. % of the total composition.

In some embodiments, the silver particles constitute at least 0.01 wt. %, at least 0.1 wt. %, at least 0.5 wt. %, at least 1 wt. %, at least 2 wt. %, at least 3 wt. %, at least 4 wt. %, at least 5 wt. % or at least 10 wt. % of the composition. In some embodiments, the silver particles constitute at most 10 wt. %, at most 5 wt. %, at most 4 wt. %, at most 3 wt. %, at most 2 wt. %, at most 1 wt. %, at most 0.5 wt. %, or at most 0.1 wt. % of the composition.

In some embodiments of the composition, use or method disclosed herein, the UV-protective composition is a composition for human or animal use, formulated as a topical composition. The topical composition may optionally be provided in a form selected from the group consisting of a cream, an emulsion, a gel, a lotion, a mousse, a paste and a spray. If desired, the topical composition can also be formulated into make-up cosmetics, for example, foundation, blusher, etc.

In some embodiments, the topical composition further comprises a dermatologically or cosmetically or pharmaceutically acceptable carrier.

In some embodiments, the topical composition further comprises one or more dermatologically or cosmetically or pharmaceutically acceptable additives or excipients, such as colorants, preservatives, fragrances, humectants, emollients, emulsifiers, waterproofing agents, surfactants, dispersants, thickeners, viscosity modifiers, anti-foaming agents, conditioning agents, antioxidants and the like. Such additives or excipients and the concentrations at which each can effectively accomplish its respective functions, are known to persons skilled in the pertinent art and need not be further detailed.

In some embodiments, the topical composition is a sunscreen composition.

In some embodiments, the UV-protective composition is in the form of a coating that can be applied to the surface of an inanimate object. The coating composition may be provided in a form selected from the group consisting of liquid coat, an emulsion, a cream, a gel, a paste and a spray.

In another aspect of the present disclosure, there is provided a method for the preparation of the compositions disclosed herein.

According to a further aspect of some embodiments of the disclosure, there is provided a UV-protective composition as disclosed herein, for use in protecting a subject, such as a human subject or a non-human animal, against a harmful effect of ultraviolet radiation, in some embodiments providing broad-spectrum protection against both ultraviolet A and ultraviolet B radiation.

In some embodiments, the composition is for use in protecting the skin of a subject, against a harmful effect of ultraviolet radiation, in some embodiments providing broad-spectrum protection against both ultraviolet A and ultraviolet B radiation.

In some embodiments, the composition is for use in protecting the hair of a subject, such as a human subject, against a harmful effect of ultraviolet radiation, in some embodiments against harmful effects of both ultraviolet A and ultraviolet B radiation.

The skin may be the skin of the face, of the arms, of the legs, of the neck of the torso, or of any other area of the body that can be exposed to UV radiation.

In some embodiments, the sunscreen composition as disclosed herein is applied to the skin of the subject prior to or during exposure to UV radiation. In some embodiments, the composition is reapplied intermittently, for example every 10 hours, every 9 hours, every 8 hours, every 7 hours, every 6 hours, every 5 hours, every 4 hours, every 3 hours, every 2 hours or every hour, or any intermediate value, during exposure to UV radiation.

In some embodiments, the UV-protective composition is for protecting the hair of a subject against ultraviolet radiation and is provided in a form selected from the group consisting of a cream, an emulsion, a gel, a lotion, a mousse, a paste and a spray. In some embodiments, the composition is provided in the form of a shampoo, a conditioner or a hair mask.

In some embodiments, the composition is formulated to be applied to the hair, or is applied to the hair, for a fixed period of time (such as up to 1 minute, up to 2 minutes, up to 3 minutes, up to 4 minutes or up to 5 minutes, up to 10 minutes, up to 15 minutes, up to 20 minutes, up to 25 minutes or up to 30 minutes) prior to rinsing. In some embodiments, the conditioner or hair mask is formulated for application to the hair, or is applied to the hair without rinsing, such that the conditioner or hair mask remains on the hair.

According to a further aspect of some embodiments of the disclosure, there is provided a UV-protective composition as disclosed herein, for use in protecting an inanimate object, against a harmful effect of ultraviolet radiation, in some embodiments providing broad-spectrum protection against both ultraviolet A and ultraviolet B radiation. In some embodiments, the UV-protective composition for use in protecting an inanimate object, is capable of protecting the object against a harmful effect of ultraviolet B radiation.

According to a further aspect of some embodiments of the disclosure, there is provided a method of protecting the skin or the hair of a subject against a harmful effect of ultraviolet radiation, the method comprising applying to the skin and/or the hair of the subject a sunscreen composition comprising a matrix comprising a polymer and a carrier (an oil-based carrier or a water-based carrier); and particles of doped or undoped BLT crystals, dispersed in the matrix.

According to a further aspect of some embodiments of the disclosure, there is provided the use of a matrix comprising a polymer and a carrier (an oil-based carrier or a water-based carrier); and particles of a UV-protective-agent comprising doped or undoped BLT crystals, dispersed in the matrix, in the manufacture of a composition for protection of the skin and/or the hair of a subject against a harmful effect of ultraviolet radiation.

According to a further aspect of some embodiments of the disclosure, there is provided the use of a matrix comprising a polymer and a carrier (an oil-based carrier or a water-based carrier); and particles of a UV-protective agent comprising doped or undoped BLT crystals, dispersed in the matrix, in the manufacture of a composition for protection of exterior surfaces of an inanimate object against a harmful effect of ultraviolet radiation. The exterior surface may comprise the surface of any porous or non-porous material, including, but not limited to glass, fabrics, leathers, woods, cardboards, metals, plastics, rubbers, ceramics and other structural materials.

The composition for the protection of inanimate objects against UV radiation, can be formulated in any form suitable for application to the surface of the inanimate object on which it is to be used.

### EXAMPLES

### Materials and Methods

### Materials

The following materials were purchased from Sigma Aldrich, USA:

| | |
|---|---|
| Bi₂O₃ (99.9% pure) | CAS 1304-76-3 |
| Fe₂O₃ (99% pure) | CAS 1309-37-1 |
| La₂O₃ (99.9% pure) | CAS 1312-81-8 |
| Poly Acrylic Acid Sodium base (PAA) | CAS 9003-04-7 |
| TiO₂ (99% pure) | CAS 13463-67-7 |

The milling media, namely Zirconia beads having an average diameter of 2 mm, were purchased from Pingxiang Lier Ceramic Co., China.

### Equipment

High Resolution Scanning Electron Microscope HSEM/TEM Magellan XHR 400L FE-SEM by Nanolab Technologies, Albany, N.Y., USA.

High Resolution X-ray diffractometer XRD Rigaku SmartLab® with Cu radiation generated at 40 kV and 30 mA (CuKa=1.542 A) as the X-ray source.

Particle Size Analyzers (Dynamic Light Scattering) Zen 3600 Zetasizer (for particles in the range of up to about 10 μm) and Mastersizer 2000 (for particles in the range of 0.02 μm to 2000 μm) by Malvern Instruments, Malvern, UK.

Oven, Vulcan-Hart 3-1750 multi-stage programmable box furnace.

Temperature controllable circulating water bath, BL-30L 9 liter 1/3HP by MRC, Hampstead, London, UK.

Grinding Mill Model HD-01 Attritor by Union Process®, Inc., Akron, Ohio, USA.

Analytical Balance XSE by Mettler-Toledo International Inc., Columbus, Ohio, USA.

Mortar Grinder Pulverisette 2 by Fritsch GmbH, Idar-Oberstein, Germany

Double Planetary Mixer by Charles Ross & Son Company, Hauppauge, N.Y., USA.

### Example 1

### Preparation of BLT Crystals

BLT crystals having the formula Bi₍₄₋ₓ₎La₍ₓ₎Ti_{(3-y)}Fe_{(y)}O₁₂ as an ultraviolet-absorbing agent, wherein x is between 0.1 and 1.5; and wherein y is between 0 and 2 were prepared by a solid solution method. The Fe-doped crystals included five different molar ratios of Fe to Ti, as follows: 0.0625:2.9375, 0.125:2.875, 0.25:2.75, 1:2 or 1.5:1.5.

In this process, the constituent metal oxides were mixed together in powder form so as to obtain the desired stoichiometric amount. Bi₂O₃, having a MW of 465.96 g/mol, La₂O₃ having a MW of 325.82 g/mol, TiO₂ having a MW of 79.87 g/mol were mixed in desired ratio so that the combined BLTO powders amounted to about 200 grams. When desired, Fe₂O₃ having a MW of 159.69 g/mol, was added while the amount of titanium dioxide was reduced, the amount of ferric oxide selected to provide the required doping ratio. The combination of metal oxides, which in case of intended iron doping can be termed the BLTO-Fe powders, amounted likewise to about 200 grams.

All materials were weighed using an analytical scale (Mettler Toledo, USA).

The powders of the constituent oxides were then mixed together for about 10 minutes at 70 rpm at ambient temperature in a Pulverisette 2 mortar grinder (Fritsch, Germany), so as to obtain homogeneously mixed powders (BLTO or BLTO-Fe, as appropriate). The mixed powders were transferred to a 500 ml alumina crucible and sintered or calcined by heating in a ceramic oven at a rate of 40° C. per minute until the temperature reached 1000° C., and maintaining at this temperature for 24 hours, allowing for the formation of the desired doped or undoped BLT crystals. It is believed that under such conditions, the iron atoms can substitute the titanium atoms in the orthorhombic structure of the BLT to provide doping without breaking the crystallographic symmetry.

After 24 hours at 1000° C., the samples were allowed to cool down to ambient temperature (circa 23° C.), at which time they were again ground to homogeneous powder for about 10 minutes at 70 rpm by the Pulverisette 2 mortar grinder.

Powders of doped or undoped BLT crystals prepared as above-described were either used or analyzed “as is” in coarse form, or further size-reduced and used and analyzed in the form of nanoparticles, as described in following examples. It is to be understood that the coarse material was manually ground with a mortar and pestle to disassociate any gross agglomerate that may be present in the resulting powders, so as to eliminate coarse lumps of particles. In bulk size, the BLT compounds displayed a pale yellow shade if undoped and a reddish tint if doped, the color intensity depending on the degree of iron doping.

### Example 2

### Crystal Structure Determination

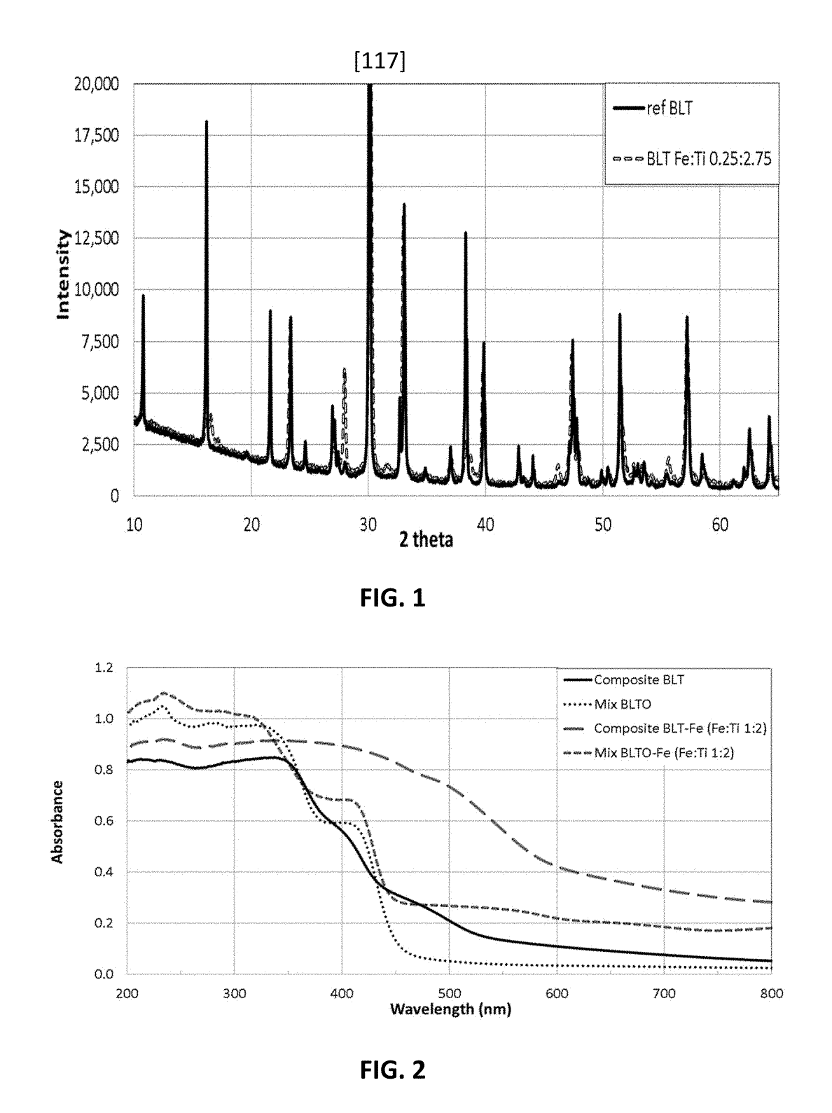

The crystal structure of doped BLT crystals for a Fe:Ti substitution of 0.25:2.75 as above-prepared was determined by powder XRD using Rigaku TTRAX-III X-ray diffractometer. The X-ray source (Cu anode) was operated at a voltage of 40 kV and a current of 30 mA on packed powder samples. Data were collected in continuous detector scan mode at a step size of 0.02°/step. Diffractograms were collected over the 2Θ range of 10° to 65°. The results are shown in FIG. 1, wherein the pattern of undoped BLT crystals is displayed as a continuous line, whereas that of Fe:Ti 0.25:2.75 doped BLT crystals is shown as a dotted line. For both materials, a predominant peak is seen around 2Θ of about 30° and doping did not significantly affect the crystalline peaks characteristic of the BLT crystals.

### Example 3

### Absorbance Determination in Powder

Absorbance correlation of coarse powders over the wavelength range of 200-800 nm was calculated using a Cary 300 UV-Vis spectrophotometer with an integrated sphere detector (Agilent Technologies, Santa Clara, Calif., USA).

Briefly, the absorbance of the samples was qualitatively estimated by subtracting the amount of light reflected from the powder sample, gathered by the integrated sphere detector of the spectrophotometer, from the amount of light reflected from a white surface (which reflects all incident light). Since the extent of penetration of the light into the samples and the extent of scattering of the sample is unknown, this measurement provides an absorbance profile of the sample rather than a true quantitative measurement.

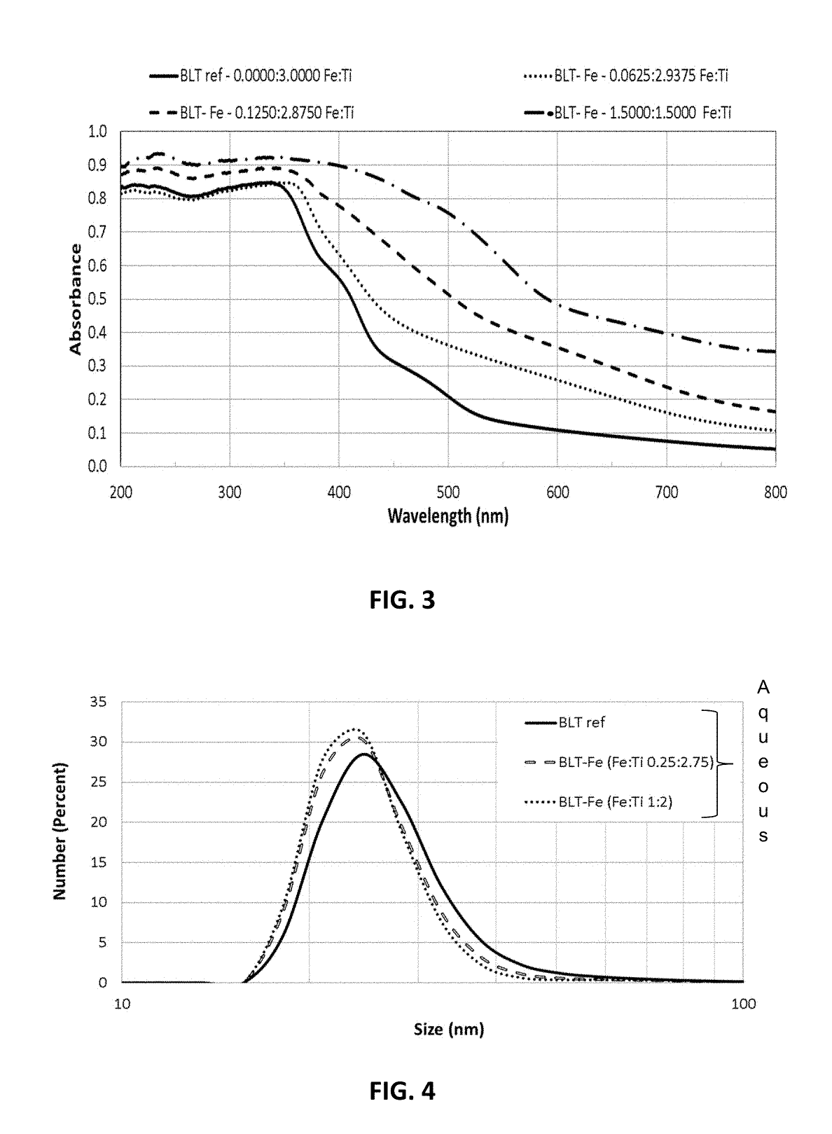

Results, showing correlation to absorbance as a function of wavelength, determined by diffuse reflection measurement gathered by the integrated sphere method, are presented in FIGS. 2 and 3.

FIG. 2 shows the absorbance of doped (Fe:Ti 1:2) or undoped BLT crystals, as obtained following the sintering method of Example 1, as compared to their respective mixture of constituent metal oxides. As seen in the figure, the sintered materials differ from the initial mix of the constituents. Whereas the constituent mixtures display “step-like” variations in absorbance, each step predominantly attributable to one or another of the individual constituents, the formed crystals display much smoother variation curves. Undoped BLT crystals show a relatively constant UV absorbance of about 0.84 from 200 nm to about 350 nm, with a relatively even decrease till about 550 nm and with a still relatively high absorbance of about 0.56 at 400 nm, this absorbance representing about 67% of the initial plateau value of about 0.84. Doped (Fe:Ti 1:2) BLT crystals, show a relatively constant UV absorbance of about 0.90 from 200 nm to about 415 nm, suggesting that the Fe-doped BLT may provide for a broader range of UV protection.

FIG. 3 shows the impact of varying degrees of doping on the absorbance of BLT crystals, all such coarse powders having been prepared according to Example 1. As seen in the figure, where only part of the doped BLT crystal samples are shown for clarity, the higher the degree of doping in the range tested, the higher the initial “plateau” absorbance and/or the broader the UV range over which such materials significantly absorb radiation. Whereas undoped BLT shows a relatively constant UV absorbance of about 0.84 from 200 nm to about 350 nm, its Fe:Ti 0.0625:2.9375 doped variant displays an approximate average absorbance of 0.82 from 200 nm to about 380 nm, whereas the Fe:Ti 0.125:2.875 doped variant displays an average absorbance of about 0.88 from 200 nm to about 380 nm and the Fe:Ti 1.5:1.5 doped variant displays an average absorbance of about 0.91 from 200 nm to about 430 nm.

### Example 4

### Preparation of Nanoparticles

Nanoparticles of doped or undoped BLT crystals, as well as their respective constituents and mixtures thereof when desired, were prepared from the ground samples obtained in Example 1 or from their stock powders. Generally, all such samples or stock powders contained particles having a size greater than about 5 micrometer (μm) and may be referred hereinafter as the coarse materials. The coarse powders were milled in an Attritor grinding mill (HD-01 by Union Process®) using a batch size of 200 g with solid loading 10% (20 g) as follows.

All materials were weighed using an analytical scale (XSE by Mettler Toledo). 20 g of PAA dispersant was weighed and dispersed in about 100 ml of deionized water. 20 g of coarse powder was weighed and introduced into the dispersant-containing liquid to provide a dispersant to inorganic material ratio of 1:1 yielding a slurry of the inorganic material. Water was added to complete batch size to 200 g, the solids constituting about 10 wt. % of the sample.

The aqueous slurry of inorganic material was then placed in a zirconia pot with 2300 g of 2 mm diameter zirconia grinding beads. The pot was placed in the grinding mill, and the grinding mill activated at 700 rpm for about 75 hours at 25° C.

The hydrodynamic diameter of the nanoparticles obtained by this method was determined by Dynamic Light Scattering, using a Zen 3600 Zetasizer from Malvern Instruments Ltd. (Malvern, UK). A sample of the milled nanoparticles was further diluted in deionized water to form a suspension having a solid concentration of about 0.5 wt. %.

Representative results, showing the percentage of number of doped and undoped BLT crystal particles having hydrodynamic diameters in the range of 1-100 nm are presented in FIG. 4.

As shown in the figure, the particles of inorganic material in suspension had hydrodynamic diameters of up to about 100 nm. The majority of doped and undoped BLT crystal particles had hydrodynamic diameters in the size range of from about 15 nm and up to about 60 nm or 50 nm. The predominant peak of undoped BLT, was around about 26 nm, whereas the two Fe-doped variants displayed similar peaks at about 28 nm for Fe:Ti 0.25:2.75 and about 24 nm for Fe:Ti 1:2 Results of the particle size distribution of the nanoparticles prepared as herein described, namely the maximum hydrodynamic diameter of a percentage of the population, are provided in the Table 1 below, in terms of percent of number of particles.

TABLE 1

| | Max. Hydrodynamic Diameter (nm) | | | | |
|---|---|---|---|---|---|
| Material | 50.0% | 90.0% | 95.0% | 97.5% | 99.0% |
| Undoped BLT | 25.6 | 37.0 | 43.1 | 53.0 | 72.0 |
| Fe:Ti 0:3 | | | | | |
| Doped BLT | 28.0 | 34.0 | 37.7 | 44.9 | 64.6 |
| Fe:Ti 0.25:2.75 | | | | | |
| Doped BLT | 24.0 | 33.0 | 36.5 | 43.0 | 68.3 |
| Fe:Ti 1:2 | | | | | |

As can be seen from the above table, at least 99% of the nanoparticles of doped or undoped BLT as prepared and size-reduced according to the present teachings have a dimension of at most 100 nm.

### Example 5

### Absorbance of Suspended Crystal Nanoparticles

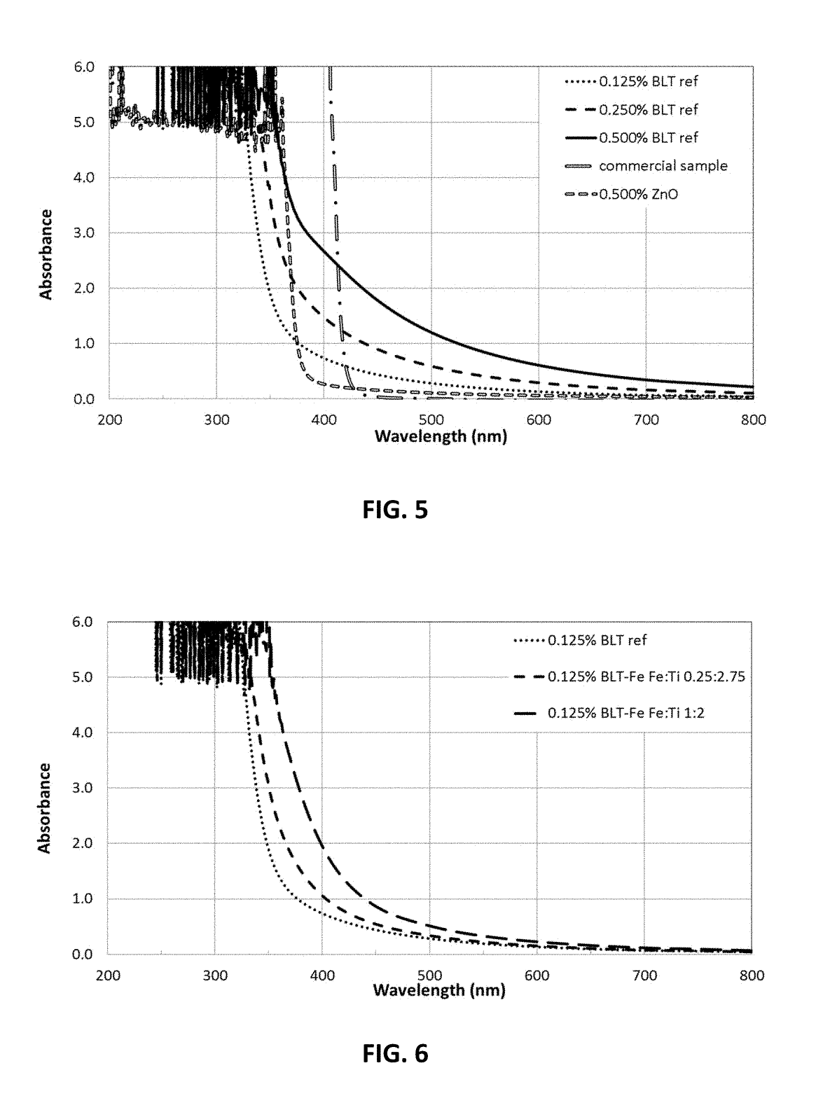

Absorbance of the doped and undoped BLT crystal nanoparticles prepared according to Example 4 was measured over the wavelength range of 200-800 nm using a Cary 300 UV-Vis spectrophotometer with quartz cuvette (10 mm light pathway). The samples were diluted in the vehicle in which the inorganic materials were milled (namely with deionized water containing 20 wt. % PAA) to provide any desired predetermined solid concentration (e.g., 0.125 wt. %, 0.25 wt. % and 0.5 wt. %,). Results are presented in FIGS. 5 and 6. For convenience, it should be recalled that an absorbance value of 1 indicates a UV blocking of at least about 90%, whereas an absorbance value of 2 indicates blocking of up to 99% of the radiation.

In FIG. 5, the absorbance in the 280-400 nm wavelength range is shown for undoped BLT nanoparticles at increasing concentrations as compared to a commercial sample (Skingard® sunscreen composition of Careline®) and to a nanoparticulated control consisting of 0.5 wt. % ZnO prepared by a similar method and having a D_{N}50 of about 25 nm. As can be seen in the figure, the control zinc oxide and commercial sample displayed a steeper drop in absorbance than the present composite material. Undoped BLT crystals displayed a very significant absorbance up to at least 400 nm at all concentrations tested. While at 400 nm the absorbance provided by 0.5 wt. % of ZnO was of only about 0.27, undoped BLT displayed at this same wavelength an absorbance of about 0.74, 1.47 and 2.66 for compositions containing solid concentrations of 0.125 wt. %, 0.25 wt. % and 0.5 wt. %, respectively. Thus, at the same 0.5 wt. % concentration as the zinc oxide control, the BLT crystals prepared according to the present teachings displayed a ten-fold higher value, which indicates a much more significant difference in absorbance efficiency. Moreover, it can be seen that increasing the concentration of BLT in the tested range, resulted in a broadening of the ranges of wavelengths wherein the composite provided radiation absorbance.

Since 0.125 wt. % of undoped BLT already provides for a significant absorbance of about 0.74 at 400 nm, the absorbance of Fe-doped BLT crystals (Fe:Ti 0.25:2.75 and Fe:Ti 1:2), is displayed in FIG. 6 only at this concentration. As can be seen in the figure, a higher level of substitution of titanium atoms being replaced by iron atoms led to absorbance over a broader spectrum and/or a higher absorbance at any particular wavelength within the range of efficiency. For instance, while 0.125 wt. % of undoped BLT provided for an absorbance of about 0.74 at 400 nm, the same concentration of Fe-doped BLT (Fe:Ti 0.25:2.75 and Fe:Ti 1:2) respectively displayed absorbance of 1.06 and 1.95.

Higher concentrations of nanoparticles of Fe-doped BLT were also tested and displayed a pattern similar to that of unsubstituted BLT, namely over the range tested a higher concentration of material led to a broader range of wavelength with efficient absorbance.

### Example 6

### Scanning Electron Microscope Studies

The doped and undoped BLT crystal nanoparticles were also studied by High Resolution Scanning Electron Microscopy (HR-SEM) using Magellan™ 400 HSEM/TEM by Nanolab Technologies.

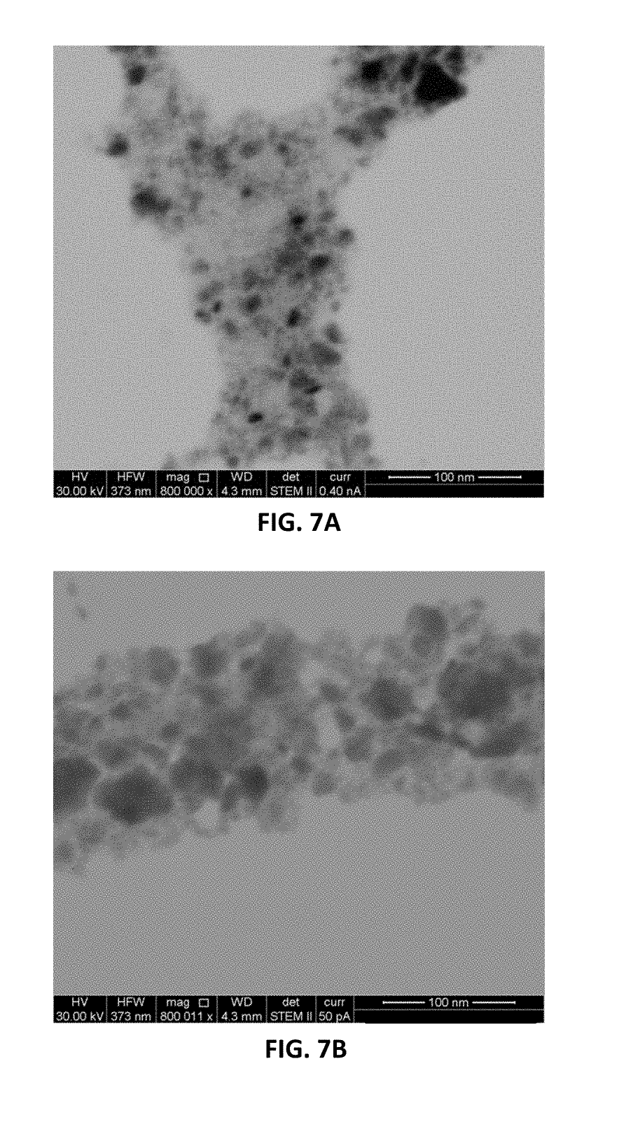

FIG. 7A shows an image for undoped BLT crystal nanoparticles, wherein FIG. 7B shows an image for Fe-doped BLT crystal nanoparticles (Fe:Ti 1:2).

### Example 7

### Determination of Critical Wavelength

Based on the absorbance spectra determined according to previous Examples, critical wavelength was calculated for undoped BLT crystals and for two Fe-doped variants, all measured at nanoparticle concentration of 0.5 wt. % and 0.125 wt. %. A suspension of nanoparticles of Zinc Oxide at 0.5 wt. % served as control.

Briefly, in order to quantify the breadth of UV protection, the absorbance of the sunscreen composition was integrated from 290 nm to 400 nm the sum reached defining 100% of the total absorbance of the sunscreen in the UV region. The wavelength at which the summed absorbance reaches 90% absorbance was determined as the ‘critical wavelength’ which provided a measure of the breadth of sunscreen protection.

The critical wavelength λ_{c} was defined according to the following equation:

$${{\int }^{{λ}_{c}}}_{290} Ig [ 1 \text{/} T ( λ ) ] d λ = 0.9 \cdot {{\int }_{290}}^{400} Ig [ 1 \text{/} T ( λ ) ] d λ$$

wherein:
λ_{c} is the critical wavelength;
T(λ) is the mean transmittance for each wavelength; and
Dλ is the wavelength interval between measurements.
Critical wavelengths as calculated are presented in Table 2 below.

TABLE 2

Critical Wavelength (nm)

| Inorganic Material | 0.125 wt. % | 0.5 wt. % |
|---|---|---|
| BLT undoped | 370 | 390 |
| BLT-Fe Fe:Ti 0.25:2.75 | 374 | 393 |
| BLT-Fe Fe:Ti 1:2 | 378 | 397 |
| ZnO Control | Not Available | 362 |

As can be seen from the above table, according to the Critical Wavelength Method, undoped and Fe-doped BLT crystal nanoparticles can be classified as providing broad spectrum protection (i.e. having a critical wavelength of 370 nm or more) at concentrations of as low as 0.125 wt. % and 0.5 wt. % Such results are superior to those achieved by the control suspension consisting of ZnO nanoparticles having similar particle size distribution when tested at the higher concentration of 0.5 wt. %.

### Example 8

### Preparation of Composition Comprising Polymer Matrix and BLT Crystals

The nanoparticles of doped or undoped BLT crystals prepared according to the present teachings and above-examples can be further processed so as to be embedded or immobilized within a polymer matrix. Suitable methods and polymers are described by the present Applicant in PCT Publication No. WO 2017/013633, incorporated herein by reference in its entirety as if fully set forth herein. In particular, Example 2 of the reference provides for the preparation of a polymer matrix, whereas Example 3 teaches how to blend such matrix with nanoparticles, and how to further process such mixture so as to obtain polymer embedded particles.

### Example 9

### Preparation of Composition Comprising BLT Crystals in Wood Lacquer

Doped and undoped BLT crystal nanoparticles are diluted in a clear wood lacquer (Tambour Clear Glossy Lacquer for Wood No. 8, Cat. No. 149-001) to a particle concentration of 1% by weight of the total lacquer composition. The resulting mixtures are sonicated for 30 seconds using a Misonix Sonicator tip (Misonix, Inc.) at amplitude 100, 15 W. The sonicated lacquer dispersions are applied upon a microscopic glass slide at an initial thickness of about 100 μm (using 100 μm thick spacers and a leveling rod). The lacquer coated slides are left to dry for at least 12 hours at ambient temperature (circa 23° C.) resulting in a dried layer of sample of about 5 μm. The lacquer devoid of added nanoparticles serves as control. Absorbance of the dried layers of lacquer over the wavelength range of 200-800 nm is assessed using a Cary 300 UV-Vis spectrophotometer.

### Example 10

### Non-Aqueous Compositions Comprising Doped BLT Crystals

The Fe doped BLT crystals were prepared as described in Examples 1 to 3.

### Preparation of Nanoparticles

Nanoparticles of doped BLT crystals were prepared from the ground samples obtained in Example 1. Generally, all such samples contained particles having a size greater than about 5 micrometer (μm) and may be referred hereinafter as the coarse materials. The coarse powders were milled in an Attritor grinding mill (HD-01 by Union Process®) using a batch size of 300 g with solid loading 10% (30 g) as follows.

All materials were weighed using an analytical scale (XSE by Mettler Toledo). 30 g of polyhydroxystearic acid (commercially available from Innospec Performance Chemicals as Dispersun DSP-OL100 or Dispersun DSP-OL300) dispersant was weighed and dispersed in about 100 ml of Isopar™ L of ExxonMobil Chemicals or C12-C15 alkyl benzoate (commercially available from Phoenix Chemical as Pelemol® 256). 30 g of coarse powder of Fe-doped BLT was weighed and introduced into the dispersant-containing liquid to provide a dispersant to inorganic material weight per weight ratio of 1:1 yielding a slurry of the inorganic material. Isopar™ L or C12-C15 alkyl benzoate was added to complete batch size to 300 g, the solids constituting about 10 wt. % of the sample.

The oily slurry of inorganic material was then placed in a zirconia pot with 2300 g of 2 mm diameter zirconia grinding beads. The pot was placed in the grinding mill, and the grinding mill activated at 700 rpm for about 75 hours at 25° C.

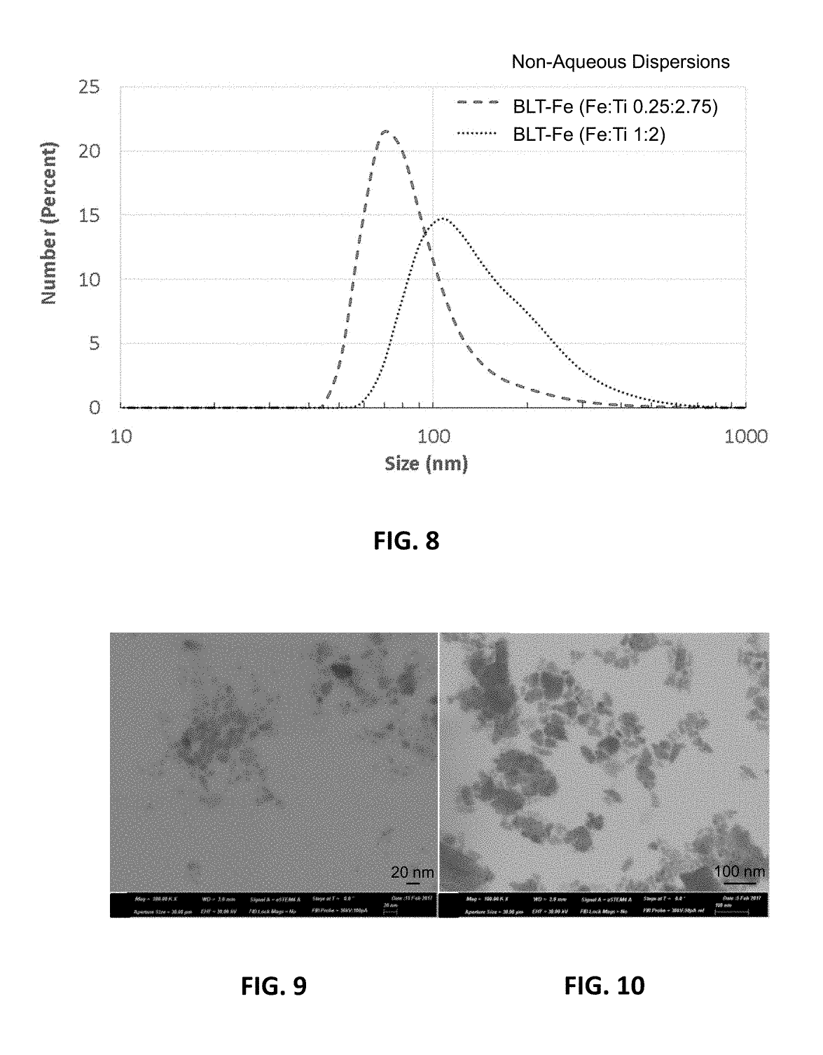

The hydrodynamic diameter of the milled particles was determined by Dynamic Light Scattering, using a Malvern Nano ZS Zetasizer particle size analyzer. A sample of the milled nanoparticles was further diluted in Isopar® L to form a suspension having a solid inorganic concentration of about 0.1 wt. % for the sake of such measurements. Representative results, showing the hydrodynamic diameters of Fe-doped BLT particles, having Fe:Ti doping of 1:2 or 0.25:2.75, expressed in terms of percentage of number of particles in the range of 10-1,000 nm are presented in FIG. 8. The sample including the BLT doped at Fe:Ti of 1:2 is represented by the dispersion prepared using DSP-OL300 in Isopar® L, while the sample including the BLT doped at Fe:Ti of 0.25:2.75 is represented by the dispersion prepared using DSP-OL100 in C12-C15 alkyl benzoate. Other dispersions using the alternative combinations of dispersants and non-aqueous/oily carriers gave similar distributions. No peaks were observed outside the presented range.

As shown in FIG. 8, the milled particles of solid inorganic crystals in non-aqueous suspensions had hydrodynamic diameters of up to about 500 nm. The majority of BLT nanoparticles Fe:Ti doped at 0.25:2.75 had hydrodynamic diameters in the size range of from about 40 nm and up to about 300 nm, with a predominant peak around about 70 nm. The majority of BLT particles Fe:Ti doped at 1:2 had hydrodynamic diameters in the size range of from about 60 nm and up to about 500 nm, with a predominant peak around about 110 nm. Results of the particle size distribution of the nanoparticles prepared as herein described, namely the maximum hydrodynamic diameter of a percentage of the population, are provided in the Table 3 below, in terms of percent of number of particles.

TABLE 3

| | Max. Hydrodynamic Diameter | | |
|---|---|---|---|
| Material | D_{N}10 | D_{N}50 | D_{N}90 |
| BLT 1:10 | 58.0 nm | 79.5 nm | 138.0 nm |
| BLT 1:2 | 84.3 nm | 128.0 nm | 256.0 nm |

As the above dynamic light scattering measurements, which assume, for the sake of hydrodynamic diameter calculations, that the particles are perfect spheres tend to overestimate the actual size of the particles, in particular if non-spherical, the size of the particles of Fe-doped BLT was further assessed by STEM microscopy. FIG. 9 and FIG. 10 are STEM images of particles of Fe-doped BLT, having a Fe:Ti doping ratio of 1:2 and 0.25:2.75, respectively. It can be seen from the images that the real size of the nanoparticles is below 100 nm for both types of Fe-doped BLT nanoparticles in the non-aqueous dispersions.

### Example 11

### Preparation of Composition Comprising Swelled Polymer Matrix Macroparticles and Nanoparticles UV-Protective Agent

2 weight portions of a swelled polymer matrix (consisting of Nucrel® 699 and Isopar™ L), prepared as described in Example 3 of WO 2017/013633 to the same Applicant, were mixed with 1 weight portion of non-aqueous dispersions containing 10 wt. % inorganic nanoparticles of UV-protective agents, Fe-doped BLT having a Fe:Ti ratio of 1:2 or 0.25:2.75, prepared as described in Example 10. The oil dispersions used herein are those which served for the measurements illustrated in FIG. 8. 60-80 g Isopar™ L were added to the mixture of swelled polymer matrix and oil dispersed inorganic nanoparticles of Fe-doped BLT to give a final weight of 200 g.

200 g of the resulting mixture were placed in a zirconia pot, 2,500 g of zirconia beads of about 2.38 mm ( 3/32″) diameter were added to the pot, and the pot was placed in the grinding mill. The temperature of the pot was maintained at 25° C. while the grinding mill was set to mill the contents of the pot at 700 rpm for 12 hours resulting in a composition according to the teachings herein comprising inorganic nanoparticles of UV-protective agent dispersed and embedded in the swelled polymer matrix macroparticles.

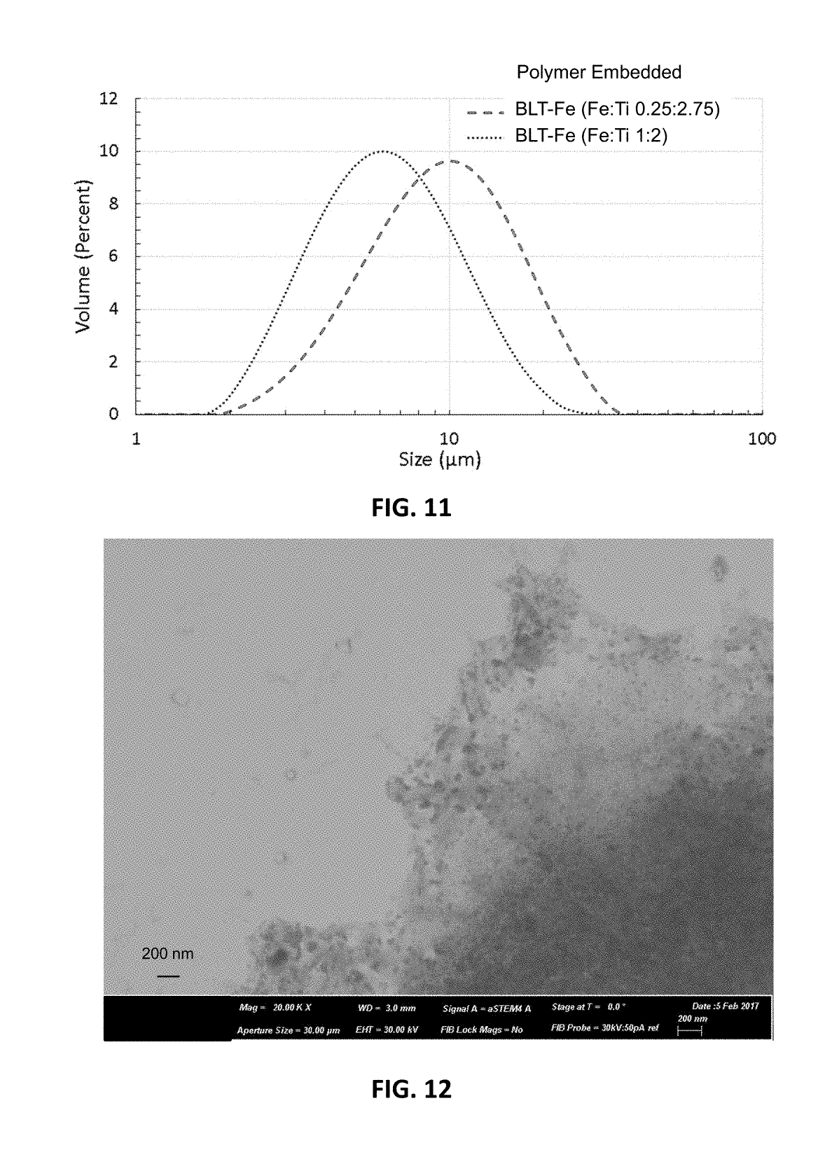

The hydrodynamic diameters of the resulting macroparticles of swelled polymer matrix were determined using Malvern Mastersizer 2000. The percentage (per volume) of macroparticles of polymer embedding the BLT-Fe doped nanoparticles are presented in FIG. 11 in the range of 1-100 μm. No peaks were observed outside of the presented range. STEM microscopic analysis performed using HR-SEM confirmed that the UV-protective nanoparticles of Fe-doped BLT were incorporated inside the polymeric macroparticles, as can be seen in FIG. 12 illustrating the embedment of BLT nanoparticles Fe-doped at a Fe:Ti ratio of 0.25:2.75, resulting in discrete, individual nanoparticles within the polymeric matrix.

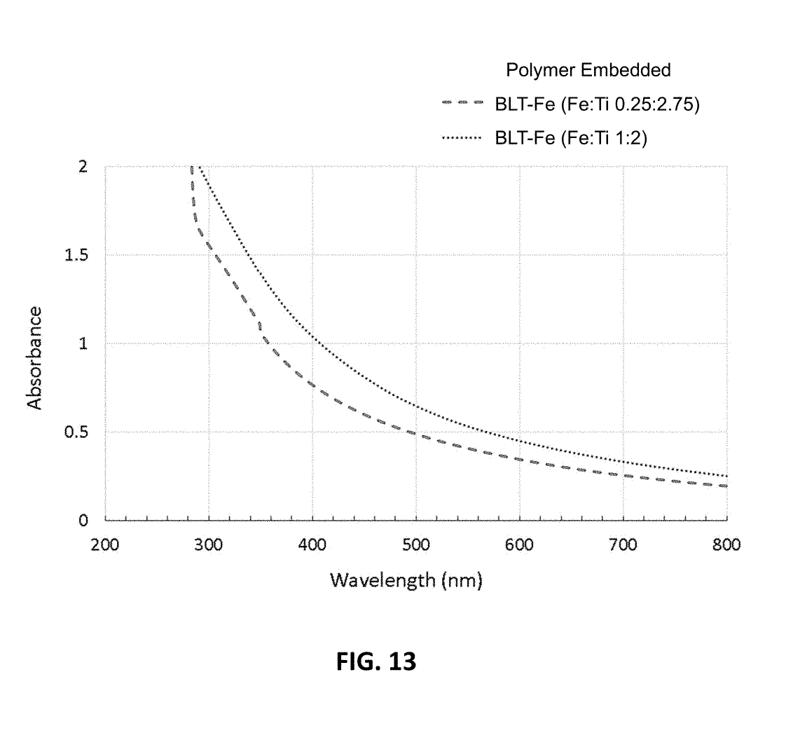

The absorbance of the non-aqueous dispersions of polymer embedded Fe-doped BLT composite lotions was measured as described in Example 3 with the following modifications. The dispersions were spread between two quartz slides (76.2×25.4×1.0 mm) and their absorbance over the wavelength range of 200-800 nm was assessed using a Cary 300 UV-Vis spectrophotometer. The results are presented in FIG. 13. Both dispersions containing at most about 2 wt. % of Fe-doped BLT significantly absorbed UV in the range of up to 400 nm.

Certain marks referenced herein may be common law or registered trademarks of third parties. Use of these marks is by way of example and shall not be construed as descriptive or limit the scope of this disclosure to material associated only with such marks.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the disclosure.

1-26. (canceled)

27. A UV-protective composition comprising Fe-doped lanthanum-modified bismuth titanate (BLT) crystals each independently having the chemical formula Bi₍₄₋ₓ₎La₍ₓ₎Ti_{(3-y)}Fe_{(y)}O₁₂ as an ultraviolet-absorbing agent, wherein x is between 0.1 and 1.5; wherein y is at least 0.01 and at most 2, the BLT crystals forming discrete nanoparticles, wherein at least 50% of a total number of said discrete nanoparticles have at least one dimension of up to 250 nm.

28. The composition according to claim 27, wherein x is between 0.5 and 1.0.

29. The composition according to claim 27, wherein y is at least 0.2 and at most 1.8.

30. The composition according to claim 27, wherein a molar ratio of Fe to Ti is 1 to 2.

31. The composition according to claim 27, wherein the Fe-doped BLT crystals are dispersed in a dispersant.

32. The composition according to claim 31, wherein the dispersant is selected from: polyacrylic acid and salts thereof; polyhydroxystearic acid; oleic acid; octyldodecyl/PPG-3myristyl ether dimer dilinoleate; butylphthalimide combined with isoproplylphthalimide; C₁₂₋₁₅ alkyl ethylhexanoate; cetyl esters; isononyl isononanoate combined with ethylhexyl isonononoate; C₁₂₋₁₅ alkyl benzoate; ethylhexyl isononanoate; polyglyceryl-3 behenate; ethyl isonanoate combined with cetyl dimethicone; propanediol dicaprylate/caprate combined with diisostearyl malate; PPG-26 dimer dilinoleate copolymer combined with isononyl isononanoate and with ethylhexyl isononanoate; dimer dilinoleyl dimer dilinoleate; diethylhexyl adipate; decyl oleate; dipentaerythrityl tetrahydroxy-stearate/tetraisostearate; octyldodecyl erucate; glyceryl ester; tribehenin; trihydroxystearin; triisostearin; triethylhexanoin; isocetyl behenate; isononyl isonanoate; isostearyl ester; triisostearin/glyceryl behenate; methyl acetyl ricinoleate; neopentylglycol dicaprate/dicaprylate; oleyl lactate; ethylhexyl pelargonate; pentaerylthrityl tetraisononanoate; propanediol dicaprylate/caprate; polyglycerol-10 hexaoleate combined with polyglyceryl-6 polyricinoleate; pentaerythrityl ester; cetearyl ethylhexanoate; tridecyl enucate; tribeherin combined with caprylic/capric triglyceride; dimer dilinoelyl dimer dilinoleate combined with triisostearin; trimethylolpropane ester; and trioctyldodecyl citrate.

33. The composition according to claim 27, wherein said discrete nanoparticles of said Fe-doped BLT crystals are dispersed with a dispersant in a polymer matrix, the polymer matrix comprising a thermoplastic polymer swelled with a carrier liquid.

34. The composition according to claim 33, wherein said polymer matrix is in the form of polymer matrix flakes wherein each flake of said polymer matrix flakes has a flake length (Lf), a flake width (Wf), and a flake thickness (Tf), said polymer matrix flakes having a dimensionless flake aspect ratio (Rf) defined by: Rf=(Lf·Wf)/(Tf)², wherein, with respect to a representative group of at least ten polymer matrix flakes, an average Rf is at least 5.

35. The composition according to claim 33, wherein the dispersant adapted to disperse the discrete nanoparticles of BLT crystals within said polymer matrix has a hydrophilic-lipophilic balance (HLB) value of at most 9.

36. The composition according to claim 33, wherein the thermoplastic polymer in the polymer matrix comprises at least one ethylene-acrylic (EAA) polymer, ethylene-methacrylic (EMMA) polymer, ethyl vinyl acetate (EVA) polymer, or combinations thereof.

37. The composition according to claim 27, formulated as one of the following: (a) a skin-care composition for application to human or non-human animal skin; (b) a hair-care composition for application to human or non-human animal hair; or (c) a coating composition for application to an inanimate surface.

38. The composition according to claim 27, for use in protecting an inanimate object against a harmful effect of ultraviolet radiation.

39. The composition according to claim 27, for use in protecting a subject, or skin or hair thereof, against a harmful effect of ultraviolet radiation.

40. The composition according to claim 38, wherein said protecting against said harmful effect of ultraviolet radiation comprises protecting against ultraviolet A radiation and ultraviolet B radiation.

41. The composition according to claim 27, wherein the composition has a critical wavelength of at least 370 nm.

42. The composition according to claim 27, wherein the composition has a critical wavelength of at most 400 nm.

43. The composition according to claim 27, wherein the composition has a critical wavelength in the range of 370 nm and 400 nm.

44. The composition according to claim 27, wherein an area under the curve (AUC) formed by the UV-absorption of the Fe-doped BLT crystals as a function of wavelength in the range of 280 nm to 400 nm (AUC₂₈₀₋₄₀₀) is at least 75% of the AUC formed by the same crystals at the same concentration in the range of 280 nm to 700 nm (AUC₂₈₀₋₇₀₀).

45. An article coated with the composition according to claim 27.
